SEQUENCE LISTING (13 sequences)

SEQ ID NO: 1 - DNA, 20 bp, synthetic construct
  source 1..20: mol_type = other DNA; note = P5 amplification primer
aatgatacggcgaccaccga

SEQ ID NO: 2 - DNA, 21 bp, synthetic construct
  source 1..21: mol_type = other DNA; note = P7 amplification primer
caagcagaagacggcatacga

SEQ ID NO: 3 - DNA, 20 bp, synthetic construct
  source 1..20: mol_type = other DNA; note = complementary P5 adapter
tcggtggtcgccgtatcatt

SEQ ID NO: 4 - DNA, 21 bp, synthetic construct
  source 1..21: mol_type = other DNA; note = complementary P7 adapter
tcgtatgccgtcttctgcttg

SEQ ID NO: 5 - DNA, 24 bp, synthetic construct
  source 1..24: mol_type = other DNA; note = PX primer
aggaggaggaggaggaggaggagg

SEQ ID NO: 6 - DNA, 30 bp, synthetic construct
  source 1..30: mol_type = other DNA; note = PY primer
gaagaagaagaagaagaagaagaagaagaa

SEQ ID NO: 7 - DNA, 24 bp, synthetic construct
  source 1..24: mol_type = other DNA; note = cPX primer
cctcctcctcctcctcctcctcct

SEQ ID NO: 8 - DNA, 30 bp, synthetic construct
  source 1..30: mol_type = other DNA; note = cPY primer
ttcttcttcttcttcttcttcttcttcttc

SEQ ID NO: 9 - protein, 13 aa, synthetic construct
  source 1..13: note = SpyTag: amino acid sequence
AHIVMVDAYKPTK

SEQ ID NO: 10 - protein, 134 aa, synthetic construct
  source 1..134: note = SpyCatcher amino acid sequence
MKGSSHHHHHHVDIPTTENLYFQGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATK

SEQ ID NO: 11 - protein, 5 aa, synthetic construct
  source 1..5: note = Sortase-coupling
LPXTG

SEQ ID NO: 12 - protein, 4 aa, synthetic construct
  source 1..4: note = Sortase-coupling
GGGG

SEQ ID NO: 13 - protein, 5 aa, synthetic construct
  source 1..5: note = Sortase-coupling
GGGGG

(19) United States
(12) Patent Application Publication
(10) Pub. No.: US 2025/0109428 A1
(43) Pub. Date: Apr. 3, 2025

(54) CAPTURING AND AMPLIFYING POLYNUCLEOTIDES USING HYBRID PARTICLES

(71) Applicant: Illumina, Inc., San Diego, CA (US)

(72) Inventors: Olivia Ghazinejad, San Diego, CA (US); Fei Shen, San Diego, CA (US); Kyle Bentz, San Diego, CA (US); Rohit Subramanian, San Diego, CA (US); Eric Brustad, San Diego, CA (US); Lena Storms, San Diego, CA (US); Xiaoyu Ma, San Diego, CA (US); Mathieu Lessard-Viger, San Diego, CA (US); Jeffrey Brodin, San Diego, CA (US)

(73) Assignee: Illumina, Inc., San Diego, CA (US)

(21) Appl. No.: 18/899,460

(22) Filed: Sep. 27, 2024

Related U.S. Application Data
(60) Provisional application No. 63/587,015, filed on Sep. 29, 2023.

(51) Int. Cl. C12Q 1/6806; B01L 3/00; C12Q 1/6844

(52) U.S. Cl. CPC C12Q 1/6806; B01L 3/5025; C12Q 1/6844; B01L 2300/0829

(57) ABSTRACT

In some examples, a hybrid particle for use in capturing a polynucleotide may include a scaffold molecule including a seeding primer and a plurality of first moieties, and a nanoparticle including a plurality of second moieties. The scaffold molecule is coupled to the nanoparticle via interactions between the plurality of first moieties and the plurality of second moieties.

### CROSS-REFERENCE TO RELATED APPLICATIONS

This application claims the benefit of U.S. Provisional Patent Application No. 63/587,015, filed Sep. 29, 2023 and entitled “Capturing and Amplifying Polynucleotides Using Hybrid Particles,” the entire contents of which are incorporated by reference herein.

### INCORPORATION BY REFERENCE OF SEQUENCE LISTING

The material in the accompanying sequence listing is hereby incorporated by reference into the application. The accompanying sequence listing XML file, named “IP-2680-US.xml”, was created on Sep. 26, 2024 and is 16 kB in size.

### BACKGROUND

Cluster amplification is an approach to amplifying polynucleotides, for example for use in genetic sequencing. Target polynucleotides are captured by primers (e.g., P5 and P7 primers) coupled to a substrate surface in a flow cell, and form “seeds” at random locations on the surface. Cycles of amplification are performed to form clusters on the surface around each seed. The clusters include copies, and complementary copies, of the seed polynucleotides. In some circumstances, the substrate is patterned so as to define regions that bound different clusters, such as wells that may be filled with respective clusters.

### SUMMARY

Examples provided herein are related to capturing and amplifying polynucleotides using hybrid particles. Methods for preparing hybrid particles also are disclosed.

Some examples herein provide a hybrid particle for use in capturing a first polynucleotide. The hybrid particle may include a scaffold molecule including a seeding primer and a plurality of first moieties. The hybrid particle also may include a nanoparticle including a plurality of second moieties. The scaffold molecule may be coupled to the nanoparticle via interactions between the plurality of first moieties and the plurality of second moieties.

In some examples, the scaffold molecule includes a dendritic molecule including: a dendritic core to which the seeding primer is coupled; and a plurality of dendrons. In some examples, each of the dendrons includes an elongated polymer. In some examples, the elongated polymer includes a second polynucleotide. In some examples, the elongated polymer includes an inert polymer.

In some examples, the scaffold molecule includes a second polynucleotide. In some examples, the second polynucleotide includes a concatemer of repeating sequences. In some examples, the concatemer of repeating sequences includes a rolling circle amplification (RCA) product. In some examples, the concatemer of repeating sequences includes expression of a plasmid.

In some examples, the scaffold molecule includes a bottlebrush molecule.

In some examples, the nanoparticle includes a silica core. In some examples, the nanoparticle includes a magnetic core.

In some examples, the nanoparticle includes a hydrogel to which the second moieties are coupled. Some examples further include a plurality of amplification primers coupled to the hydrogel. In some examples, the amplification primers include a single type of amplification primer. In some examples, the amplification primers include a mixture of different types of amplification primer.

In some examples, each of the first moieties includes a first oligonucleotide. In some examples, each of the second moieties includes a second oligonucleotide to which the first oligonucleotide is hybridized to couple the scaffold molecule to the nanoparticle. In some examples, the seeding primer is orthogonal to the second oligonucleotide. In some examples, the seeding primer is orthogonal to the first oligonucleotide.

In some examples, the first moiety and the second moiety are covalently bonded to couple the scaffold molecule to the nanoparticle.

Some examples further include the first polynucleotide, wherein the first polynucleotide includes a seeding adapter that is hybridized to the seeding primer. In some examples, the first polynucleotide is double-stranded, and the seeding adapter is single-stranded.

In some examples, the hybrid particle includes a single scaffold molecule.

Some examples herein provide a method of making a hybrid particle. The method may include suspending a plurality of nanoparticles in a solution, wherein each of the nanoparticles includes a plurality of first moieties. The method may include adding a plurality of scaffold molecules to the solution, wherein each of the scaffold molecules includes a capture primer and a plurality of second moieties. The method may include forming a first hybrid particle by coupling one of the nanoparticles to a corresponding one of the scaffold molecules via interactions between the plurality of first moieties of that nanoparticle and the plurality of second moieties of that scaffold molecule.

In some examples, the solution is located in a vessel including a region to which a plurality of complementary capture primers are coupled. The method further may include coupling the first hybrid particle to the region of the vessel via hybridization between the capture primer of the scaffold molecule of that hybrid particle and a corresponding one of the complementary capture primers. Some examples further include, while the first hybrid particle is coupled to the region of the vessel, removing at least a portion of the solution, wherein the removed portion contains nanoparticles that are not coupled to a corresponding one of the scaffold molecules and thus are not coupled to the region of the vessel. Some examples further include adding additional scaffold molecules to the removed solution, and forming another hybrid particle by coupling one of the nanoparticles in the removed solution to a corresponding one of the additional scaffold molecules via interactions between the plurality of first moieties of that nanoparticle and the plurality of second moieties of that scaffold molecule. Some examples further include, after removing at least the portion of the solution, eluting the first hybrid particle from the region of the vessel into another solution.

Some examples further include forming a second hybrid particle by coupling one of the nanoparticles to a corresponding two or more of the scaffold molecules via interactions between the plurality of first moieties of that nanoparticle and the plurality of second moieties of those scaffold molecules. Some examples further include coupling the first hybrid particle and the second hybrid particle to a surface, wherein the second hybrid particle couples to the surface with a different force than does the first hybrid particle; and using the different force to separate the first hybrid particle from the second hybrid particle.

Some examples herein provide a flowcell including a plurality of wells; and a plurality of hybrid particles. Each hybrid particle may include a scaffold molecule including a seeding primer and a plurality of first moieties; and a nanoparticle including a plurality of second moieties. The scaffold molecule may be coupled to the nanoparticle via interactions between the plurality of first moieties and the plurality of second moieties. At least some of the wells contain a single one of the hybrid particles.

In some examples, each of the hybrid particles contained within a well has a diameter which is about 60% to about 100% of a diameter of that well.

Some examples herein provide method of capturing a polynucleotide in a flowcell. The method may include flowing a plurality of hybrid particles into a flowcell including a plurality of wells. Each hybrid particle may include a scaffold molecule including a seeding primer and a plurality of first moieties; and a nanoparticle including a plurality of second moieties. The scaffold molecule may be coupled to the nanoparticle via interactions between the plurality of first moieties and the plurality of second moieties. The method may include, within at least some of the wells, respectively disposing a respective one of the hybrid particles within that well.

In some examples, the method further includes flowing a plurality of polynucleotides into the flowcell, the polynucleotides respectively including seeding adapters; and for each well in which a respective one of the hybrid particles is disposed, hybridizing a seeding adapter of one of the polynucleotides to the seeding primer of that hybrid particle.

In some examples, the method further includes, before flowing the plurality of hybrid particles into the flowcell, hybridizing seeding adapters of respective polynucleotides to seeding primers of respective hybrid particles.

In some examples, the method further includes the nanoparticle includes a hydrogel to which the second moieties are coupled. In some examples, the nanoparticle further includes a plurality of amplification primers coupled to the hydrogel. In some examples, the amplification primers include a single type of amplification primer. In some examples, the amplification primers include a mixture of different types of amplification primer.

Some examples herein provide a method of amplifying a polynucleotide. The method may include using the method of any of the aforementioned statements to capture the polynucleotide; and using the amplification primers to amplify the captured polynucleotide.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIGS. 1A-1D schematically illustrate example operations for forming a hybrid particle and capturing a polynucleotide using the hybrid particle.

FIGS. 2A-2E schematically illustrate example operations for capturing and amplifying a polynucleotide using the hybrid particle of FIG. 1B.

FIG. 3 schematically illustrates an alternative example operation for use in capturing and amplifying a polynucleotide in the operations described with reference to FIGS. 2A-2E.

FIG. 4 schematically illustrates a device including a plurality of wells in which operations such as described with reference to FIGS. 2A-2E and/or 3 are implemented.

FIGS. 5A-5D schematically illustrate different types of dendritic molecules that may be used in the present hybrid particles.

FIGS. 6A-6C schematically illustrate example hybrid particles including different types of dendritic molecules.

FIGS. 7A-7B schematically illustrate different types of polynucleotides that may be used in the present hybrid particles.

FIGS. 8A-8B schematically illustrate example hybrid particles including different types of polynucleotides.

FIG. 9 schematically illustrates an example flow of operations in a method for preparing hybrid particles.

FIGS. 10A-10B schematically illustrate another example flow of operations in a method for preparing hybrid particles.

FIGS. 11A-11B schematically additional example flows of operations in a method for preparing hybrid particles.

### DETAILED DESCRIPTION

Examples provided herein are related to capturing and amplifying polynucleotides using hybrid particles. Methods for preparing hybrid particles also are disclosed.

Monoclonality of a cluster in a given region of a sequencing flow cell is important to obtaining a sufficient signal to noise ratio (SNR) from that region to make a base call. The hybrid particles herein may be used to generate substantially monoclonal clusters through a deterministic approach. More specifically, each of the hybrid particles may include a scaffold molecule that includes a single primer for use in capturing a single target polynucleotide via hybridization. Additionally, each of the hybrid particles may include a nanoparticle to which the scaffold molecule is coupled. Before or after capturing the target polynucleotide, the hybrid particles may be inserted into a respective well of a flow cell. The nanoparticle is of similar to the size of the well into which the hybrid particle is inserted. Accordingly, once inserted into the well, the nanoparticle may sterically exclude additional hybrid particle(s) from being inserted into the well. Accordingly, among a plurality of wells having such hybrid particles therein, at least some of the wells (and potentially most, if not substantially all, of the wells) respectively may contain a single hybrid particle, and therefore may contain a single captured target polynucleotide. The target polynucleotide then may be amplified within the respective well to generate a substantially monoclonal cluster within that well.

First, some terms used herein will be briefly explained. Then, some example structures and example methods for capturing and amplifying polynucleotides using hybrid particles, and methods of preparing hybrid particles, will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The terms “substantially,” “approximately,” and “about” used throughout this specification are used to describe and account for small fluctuations, such as due to variations in processing. For example, they may refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

As used herein, “hybridize” is intended to mean noncovalently associating a first polynucleotide to a second polynucleotide along the lengths of those polymers to form a double-stranded “duplex.” For instance, two DNA polynucleotide strands may associate through complementary base pairing. The strength of the association between the first and second polynucleotides increases with the complementarity between the sequences of nucleotides within those polynucleotides. The strength of hybridization between polynucleotides may be characterized by a temperature of melting (Tm) at which 50% of the duplexes have polynucleotide strands that disassociate from one another.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar and/or phosphate moiety compared to naturally occurring nucleotides. Example modified nucleobases include inosine, xanthine, hypoxanthine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof. A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primed single stranded target polynucleotide, and can sequentially add nucleotides to the growing primer to form a “complementary copy” polynucleotide having a sequence that is complementary to that of the target polynucleotide. Another polymerase, or the same polymerase, then can form a copy of the target nucleotide by forming a complementary copy of that complementary copy polynucleotide. Any of such copies may be referred to herein as “amplicons.” DNA polymerases may bind to the target polynucleotide and then move down the target polynucleotide sequentially adding nucleotides to the free hydroxyl group at the 3′ end of a growing polynucleotide strand (growing amplicon). DNA polymerases may synthesize complementary DNA molecules from DNA templates and RNA polymerases may synthesize RNA molecules from DNA templates (transcription). Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase. Exemplary polymerases having strand displacing activity include, without limitation, the large fragment of Bst (Bacillus stearothermophilus) polymerase, exo-Klenow polymerase or sequencing grade T7 exo-polymerase. Some polymerases degrade the strand in front of them, effectively replacing it with the growing chain behind (5′ exonuclease activity). Some polymerases have an activity that degrades the strand behind them (3′ exonuclease activity). Some useful polymerases have been modified, either by mutation or otherwise, to reduce or eliminate 3′ and/or 5′ exonuclease activity.

As used herein, the term “primer” is defined as a polynucleotide to which nucleotides may be added via a free 3′ OH group. A primer may include a 3′ block preventing polymerization until the block is removed. A primer may include a modification at the 5′ terminus to allow a coupling reaction or to couple the primer to another moiety. A primer may include one or more moieties which may be cleaved under suitable conditions, such as UV light, chemistry, enzyme, or the like. The primer length may be any suitable number of bases long and may include any suitable combination of natural and non-natural nucleotides. A target polynucleotide may include an “adapter” that hybridizes to (has a sequence that is complementary to) a primer, and may be amplified so as to generate a complementary copy polynucleotide by adding nucleotides to the free 3′ OH group of the primer.

A “capture primer” is intended to mean a primer that is coupled to a hybrid particle and may hybridize to an adapter of the target polynucleotide. In some cases, a capture primer that is coupled to the hybrid particle and may hybridize to another adapter of that target polynucleotide may be referred to as an “orthogonal capture primer.” The adapters may have respective sequences that are complementary to those of capture primers to which they may hybridize. A capture primer and an orthogonal capture primer may have different and independent sequences than one another. A capture primer that may be used to hybridize to an adapter of a target polynucleotide in order to couple that polynucleotide to the hybrid particle, but that may not be used to grow a complementary strand during an amplification process, may in some cases be referred to as a “seeding primer.” A capture primer that may be used to grow a complementary strand during an amplification process may in some cases be referred to as an “amplification primer.”

As used herein, the term “substrate” refers to a material used as a support for compositions described herein. Example substrate materials may include glass, silica, plastic, quartz, metal, metal oxide, organo-silicate (e.g., polyhedral organic silsesquioxanes (POSS)), polyacrylates, tantalum oxide, complementary metal oxide semiconductor (CMOS), or combinations thereof. An example of POSS can be that described in Kehagias et al., Microelectronic Engineering 86 (2009), pp. 776-778, which is incorporated by reference in its entirety. In some examples, substrates used in the present application include silica-based substrates, such as glass, fused silica, or other silica-containing material. In some examples, substrates may include silicon, silicon nitride, or silicone hydride. In some examples, substrates used in the present application include plastic materials or components such as polyethylene, polystyrene, poly(vinyl chloride), polypropylene, nylons, polyesters, polycarbonates, and poly(methyl methacrylate). Example plastics materials include poly(methyl methacrylate), polystyrene, and cyclic olefin polymer substrates. In some examples, the substrate is or includes a silica-based material or plastic material or a combination thereof. In particular examples, the substrate has at least one surface comprising glass or a silicon-based polymer. In some examples, the substrates may include a metal. In some such examples, the metal is gold. In some examples, the substrate has at least one surface comprising a metal oxide. In one example, the surface comprises a tantalum oxide or tin oxide. Acrylamides, enones, or acrylates may also be utilized as a substrate material or component. Other substrate materials may include, but are not limited to gallium arsenide, indium phosphide, aluminum, ceramics, polyimide, quartz, resins, polymers and copolymers. In some examples, the substrate and/or the substrate surface may be, or include, quartz. In some other examples, the substrate and/or the substrate surface may be, or include, semiconductor, such as GaAs or ITO. The foregoing lists are intended to be illustrative of, but not limiting to the present application. Substrates may comprise a single material or a plurality of different materials. Substrates may be composites or laminates. In some examples, the substrate comprises an organo-silicate material. Substrates may be flat, round, spherical, rod-shaped, or any other suitable shape. Substrates may be rigid or flexible. In some examples, a substrate is a bead or a flow cell.

In some examples, a substrate is or includes a particle. As used herein, a “particle” is a small localized object which exists as a discrete unit in a given medium. In detail, the term refers to microscopic particles with sizes ranging from atoms to molecules, such as nanoparticle or colloidal particle. A particle may refer to a substrate that is sufficiently small that it may be located within a fluid (such as a liquid), that is, substantially surrounded on all sides by the fluid. As such, a particle may be carried by (moved by) the fluid in which it is located, for example by flowing the fluid from one location to another location. As explained elsewhere herein, particles can have a variety of shapes. When a particle is at least partially spherical, it may be referred to as a “bead.” In some examples, a first substrate (such as a flat or patterned surface within a flow cell) may be used to support a second substrate (such as a particle).

In some examples, a substrate includes a patterned surface. A “patterned surface” refers to an arrangement of different regions in or on an exposed layer of a substrate. For example, one or more of the regions may be features where one or more capture primers are present. The features can be separated by interstitial regions where capture primers are not present. In some examples, the pattern may be an x-y format of features that are in rows and columns. In some examples, the pattern may be a repeating arrangement of features and/or interstitial regions. In some examples, the pattern may be a random arrangement of features and/or interstitial regions. In some examples, substrate includes an array of wells (depressions) in a surface. The wells may be provided by substantially vertical sidewalls. Wells may be fabricated as is generally known in the art using a variety of techniques, including, but not limited to, photolithography, stamping techniques, molding techniques and microetching techniques. As will be appreciated by those in the art, the technique used will depend on the composition and shape of the array substrate.

The features in a patterned surface of a substrate may include wells in an array of features (e.g., microwells or nanowells) on glass, silicon, plastic or other suitable material(s) with a patterned, covalently-linked hydrogel such as poly(N-(5-azidoacetamidylpentyl) acrylamide-co-acrylamide) (PAZAM). The process creates hydrogel regions used for sequencing that may be stable over sequencing runs with a large number of cycles. The covalent linking of the hydrogel to the wells may be helpful for maintaining the hydrogel in the structured features throughout the lifetime of the structured substrate during a variety of uses. However in many examples, the hydrogel need not be fully or even partially covalently linked to the wells. For example, in some conditions silane free acrylamide (SFA) may be used as the hydrogel material.

In particular examples, a structured substrate may be made by patterning a suitable material with wells (e.g. microwells or nanowells), coating the patterned material with a hydrogel material (e.g., PAZAM, SFA or chemically modified variant thereof, such as the azidolyzed version of SFA (azido-SFA)) and polishing the surface of the hydrogel coated material, for example via chemical or mechanical polishing, thereby retaining hydrogel in the wells but removing or inactivating substantially all of the hydrogel from the interstitial regions on the surface of the structured substrate between the wells.

A patterned substrate may include, for example, wells etched into a slide or chip. The pattern of the etchings and geometry of the wells may take on a variety of different shapes and sizes, and such features may be physically or functionally separable from each other. Particularly useful substrates having such structural features include patterned substrates that may select the size of solid particles such as microspheres. An exemplary patterned substrate having these characteristics is the etched substrate used in connection with BEAD ARRAY technology (Illumina, Inc., San Diego, Calif.).

In some examples, a substrate described herein forms at least part of a flow cell or is located in or coupled to a flow cell. Flow cells may include a flow chamber that is divided into a plurality of lanes or a plurality of sectors. Example flow cells and substrates for manufacture of flow cells that may be used in methods and compositions set forth herein include, but are not limited to, those commercially available from Illumina, Inc. (San Diego, CA).

As used herein, a “hydrogel” refers to a three-dimensional polymer network structure that includes polymer chains and is at least partially hydrophilic and contains water within spaces between the polymer chains. A hydrogel may include any suitable combination of hydrophilic, hydrophobic, and/or amphiphilic polymer(s), so long as the overall polymer network is hydrophilic and contains water within spaces between the polymer chains. Hydrogels include chemical hydrogels in which both the bonding to form the polymer chains, and any cross-linking between the polymer chains, is covalent; such cross-linking during hydrogel formation may be irreversible, as distinguished from the present reversible cross-linking which is performed after the hydrogel is formed. In some cases, the chemical hydrogel may include, or may consist essentially of, brush-like structures of polymer chains attached to a surface, substantially without physical or covalent crosslinks between polymer chains, or alternatively polymer chains with multiple attachment points to a surface, resulting in loops, but also lacking interchain crosslinks. Hydrogels also include physical hydrogels in which the bonding to form the polymer chains, and any cross-linking within the polymer chains, is not covalent. Nonlimiting examples of physical hydrogels include agarose and alginate.

As used herein, the “polymer chain” of a hydrogel is intended to mean those portions of the hydrogel that are polymerized with one another during the polymerization process. Polymer chains may be cross-linked to form the hydrogel. For example, cross-linkers may be added during or after the polymerization process that forms the polymer chains. Additionally, or alternatively, in some examples the polymer chains may be deposited on a substrate surface or nanoparticle that includes functional groups to which functional groups of the polymer chains become coupled. The polymer chains may be coupled to the surface, e.g., via reactions between the functional groups of the polymer chains and the functional groups at the surface, and such coupling may cross-link the polymer chains to form the hydrogel. Such cross-linking may cause the polymer chains to covalently or non-covalently attach to one another, or may occur as a result of chain entanglement during polymerization and/or attachment to a surface.

As used herein, the term “directly” when used in reference to a layer covering the surface of a substrate or a nanoparticle is intended to mean that the layer covers the substrate's or nanoparticle's surface without a significant intermediate layer, such as, e.g., an adhesive layer or a polymer layer. Layers directly covering a surface may be attached to this surface through any chemical or physical interaction, including covalent bonds or non-covalent adhesion.

As used herein, the term “immobilized” when used in reference to a polynucleotide is intended to mean direct or indirect attachment to a substrate or hybrid particle via covalent or non-covalent bond(s). In certain examples, covalent attachment may be used, or any other suitable attachment in which the polynucleotides remain stationary or attached to a substrate or hybrid particle under conditions in which it is intended to use the substrate or hybrid particle, for example, in polynucleotide amplification or sequencing. Polynucleotides to be used as capture primers or as target polynucleotides may be immobilized such that a 3′-end is available for enzymatic extension and at least a portion of the sequence is capable of hybridizing to a complementary sequence. Immobilization may occur via hybridization to a surface attached oligonucleotide, in which case the immobilized oligonucleotide or polynucleotide may be in the 3′-5′ orientation. Alternatively, immobilization may occur by means other than base-pairing hybridization, such as covalent attachment.

As used herein, the term “array” refers to a population of substrate regions that may be differentiated from each other according to relative location. Different hybrid particles and/or different molecules (such as polynucleotides) that are at different regions of an array may be differentiated from each other according to the locations of the regions in the array. Illustratively, an individual region of an array may include one or more molecules of a particular type. For example, a substrate region may include a single hybrid particle which is coupled to a single target polynucleotide having a particular sequence, or may be coupled to a plurality of amplicons having the same sequence as one another. The regions of an array respectively may include different features than one another on the same substrate. Exemplary features include without limitation, wells in a substrate, hybrid particles in or on a substrate, projections from a substrate, ridges on a substrate or channels in a substrate. The regions of an array respectively may include different regions on different substrates than each other.

As used herein, the term “plurality” is intended to mean a population of two or more different members. Pluralities may range in size from small, medium, large, to very large. The size of small plurality may range, for example, from a few members to tens of members. Medium sized pluralities may range, for example, from tens of members to about 100 members or hundreds of members. Large pluralities may range, for example, from about hundreds of members to about 1000 members, to thousands of members and up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above exemplary ranges. Exemplary polynucleotide pluralities include, for example, populations of about 1×10⁵ or more, 5×10⁵ or more, or 1×10⁶ or more different polynucleotides. Accordingly, the definition of the term is intended to include all integer values greater than two. An upper limit of a plurality may be set, for example, by the theoretical diversity of polynucleotide sequences in a sample.

As used herein, the term “double-stranded,” when used in reference to a polynucleotide, is intended to mean that all or substantially all of the nucleotides in the polynucleotide are hydrogen bonded to respective nucleotides in a complementary polynucleotide.

As used herein, the term “single-stranded,” when used in reference to a polynucleotide, means that essentially none of the nucleotides in the polynucleotide are hydrogen bonded to a respective nucleotide in a complementary polynucleotide. A polynucleotide that has an “inability” to hybridize to another polynucleotide may be single-stranded, though not all single-stranded polynucleotides are unable to hybridize to certain other polynucleotides.

As used herein, the term “target polynucleotide” is intended to mean a polynucleotide that is the object of an analysis or action. The analysis or action includes subjecting the polynucleotide to amplification, sequencing and/or other procedure. A target polynucleotide may include nucleotide sequences additional to a target sequence to be analyzed. For example, a target polynucleotide may include one or more adapters, including an adapter that functions as a primer binding site, that flank(s) a target polynucleotide sequence that is to be analyzed. A target polynucleotide hybridized to a capture primer may include nucleotides that extend beyond the 5′ or 3′ end of the capture oligonucleotide in such a way that not all of the target polynucleotide is amenable to extension. In particular examples, target polynucleotides may have different sequences than one another but may have first and second adapters that are the same as one another. The two adapters that may flank a particular target polynucleotide sequence may have the same sequence as one another, or complementary sequences to one another, or the two adapters may have different sequences. Thus, species in a plurality of target polynucleotides may include regions of known sequence that flank regions of unknown sequence that are to be evaluated by, for example, sequencing (e.g., SBS). In some examples, target polynucleotides carry an adapter at a single end, and such adapter may be located at either the 3′ end or the 5′ end the target polynucleotide. Target polynucleotides may be used without any adapter, in which case a primer binding sequence may come directly from a sequence found in the target polynucleotide.

The terms “polynucleotide” and “oligonucleotide” are used interchangeably herein. The different terms are not intended to denote any particular difference in size, sequence, or other property unless specifically indicated otherwise. For clarity of description the terms may be used to distinguish one species of polynucleotide from another when describing a particular method or composition that includes several polynucleotide species.

As used herein, the term “amplicon,” when used in reference to a polynucleotide, is intended to means a product of copying the polynucleotide, wherein the product has a nucleotide sequence that is substantially the same as, or is substantially complementary to, at least a portion of the nucleotide sequence of the polynucleotide. “Amplification” and “amplifying” refer to the process of making an amplicon of a polynucleotide. A first amplicon of a target polynucleotide may be a complementary copy. Additional amplicons are copies that are created, after generation of the first amplicon, from the target polynucleotide or from the first amplicon. A subsequent amplicon may have a sequence that is substantially complementary to the target polynucleotide or is substantially identical to the target polynucleotide. It will be understood that a small number of mutations (e.g., due to amplification artifacts) of a polynucleotide may occur when generating an amplicon of that polynucleotide.

A substrate region or hybrid particle that includes substantially only amplicons of a given polynucleotide may be referred to as “monoclonal,” while a substrate region or hybrid particle that includes amplicons of polynucleotides having different sequences than one another may be referred to as “polyclonal.” A substrate region or hybrid particle that includes a sufficient number of amplicons of a given polynucleotide to be used to sequence that polynucleotide maybe referred to as “functionally monoclonal.” Illustratively a substrate region or hybrid particle in which about 60% or greater of the amplicons are of a given polynucleotide may be considered to be “functionally monoclonal.” Additionally, or alternatively, a substrate region or hybrid particle from which about 60% or more of a signal is from amplicons of a given polynucleotide may be considered to be “functionally monoclonal.” A polyclonal region of a substrate or hybrid particle may include different subregions therein that respectively are monoclonal. Each such monoclonal region, whether within a larger polyclonal region or on its own, may correspond to a “cluster” generated from a “seed.” The “seed” may refer to a single target polynucleotide, while the “cluster” may refer to a collection of amplicons of that target polynucleotide.

As used herein, a “scaffold molecule” is intended to refer to a molecule that includes a single seeding primer and at least one moiety that can be used to couple the molecule to another element, e.g., via one or more covalent bonds, or one or more non-covalent bonds, or a combination of covalent and non-covalent bonds. Non-limiting examples of scaffold molecules include dendritic molecules, polynucleotides that are concatamers of repeating sequences (e.g., may be or include a rolling circle amplification product or expression of a plasmid), or bottlebrush molecules.

As used herein, a “dendritic molecule” is intended to refer to a molecule in which at least some of the atoms are arranged in multiple branches, or “dendrons,” which extend from a central region, or “dendritic core” (which may be referred to herein as a “core” for simplicity). A molecule including such features may be understood to be “dendritic,” even if not specifically referred to as a “dendritic molecule.” The core of a dendritic molecule may be branched. In some examples, a core of a dendritic molecule may include a “dendritic polyamide” which is intended to refer to a branched structure including amide bonds and to which elongated polymers may be coupled so as to form dendrons. A nonlimiting example of a dendritic polyamide is a “dendritic polypeptide,” which is intended to refer to a branched polypeptide to which elongated polymers may be coupled so as to form dendrons. In some examples, a capture primer (such as a seeding primer or amplification primer) may be coupled to the dendritic core and may be used to capture a polynucleotide having an adapter which is substantially complementary to the capture primer of the dendritic molecule.

A “bottlebrush molecule” is intended to refer to a type of dendritic molecule in which the dendritic core is elongated and unbranched, and different unbranched dendrons extend outwardly from the dendritic core.

As used herein, a “hybrid particle” is intended to refer to a particle that includes both a scaffold molecule and a nanoparticle. The scaffold molecule may be coupled to the nanoparticle via one or more covalent bonds, or one or more non-covalent bonds, or a combination of covalent and non-covalent bonds.

As used herein, a “nanoparticle” for use in a hybrid particle is made up of a plurality of atoms or molecules that move together as a unit in a fluid. In some examples, the largest dimension of a nanoparticle may be about 1 nm to about 2 μm, e.g., about 100 nm to about 500 nm. In some examples, the nanoparticle may be substantially spherical, in which case the largest dimension of the nanoparticle may be its diameter. However, nanoparticles may have any suitable shape, such as oval, rod, dumb-bell, or the like. Non-limiting examples of materials that are suitable for use in the present nanoparticles include polymer, silica, magnetic materials, metal, and the like, or any suitable combination of two or more materials. In some examples, the present nanoparticles have a magnetic or super-magnetic core, which may facilitate their manipulation.

### Structures and Methods for Capturing and Amplifying Polynucleotides Using Hybrid Particles

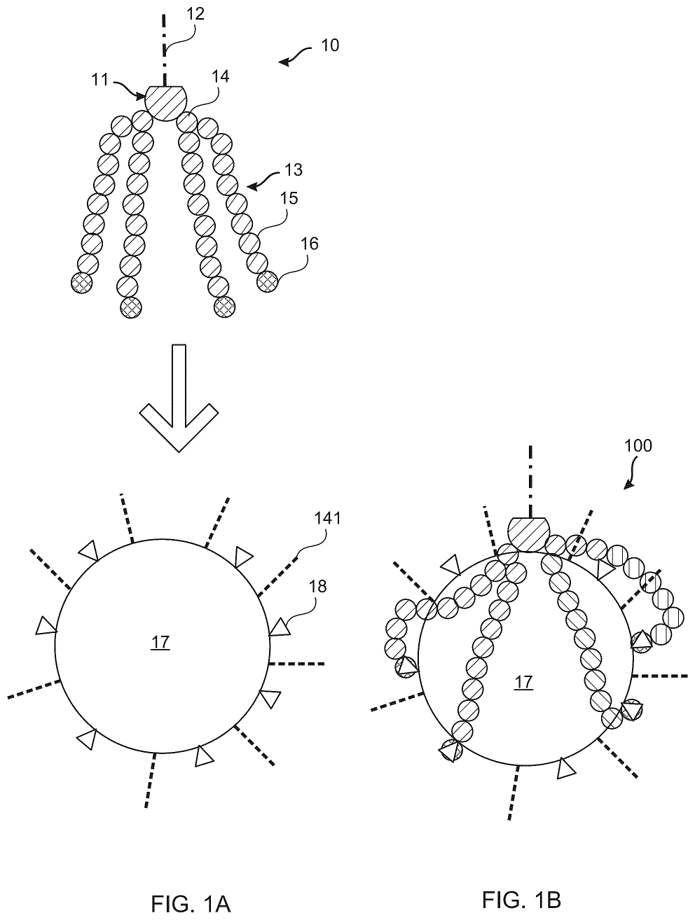

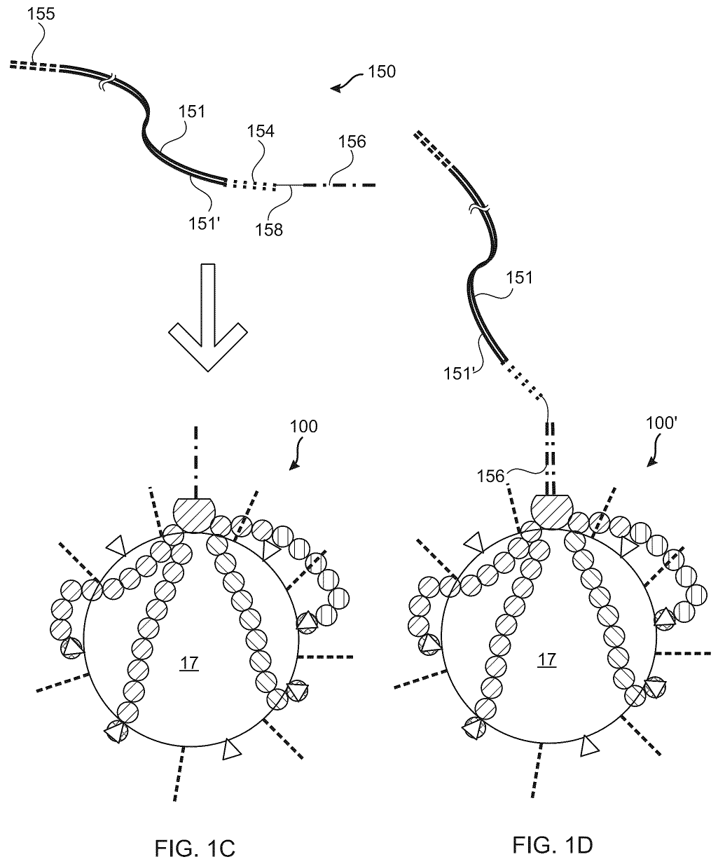

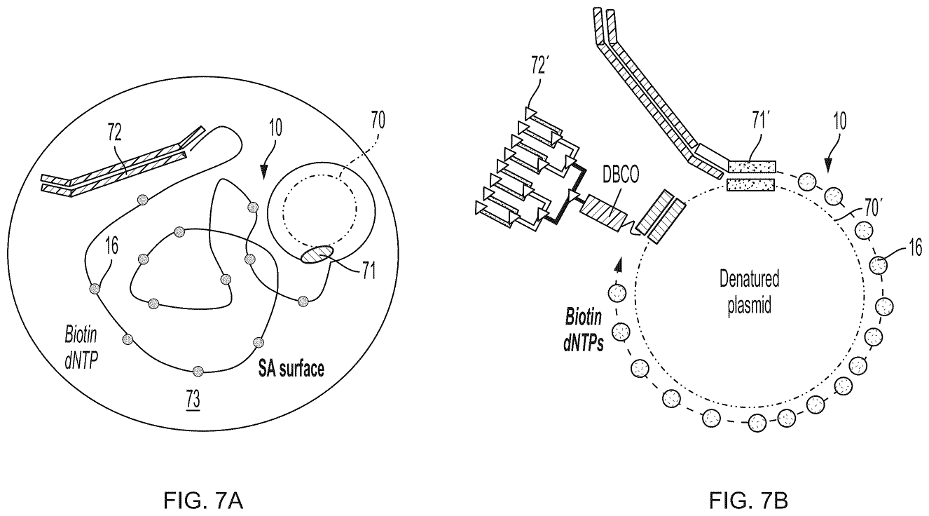

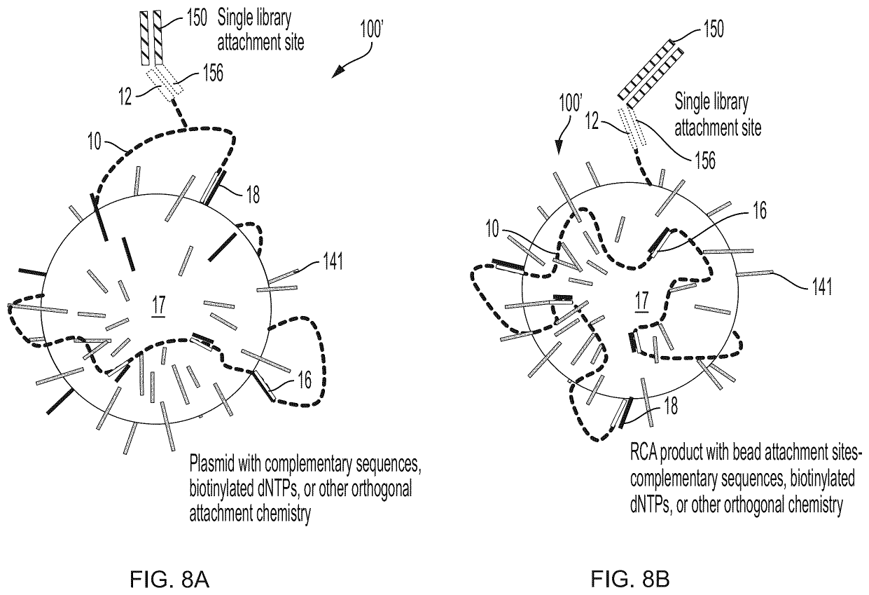

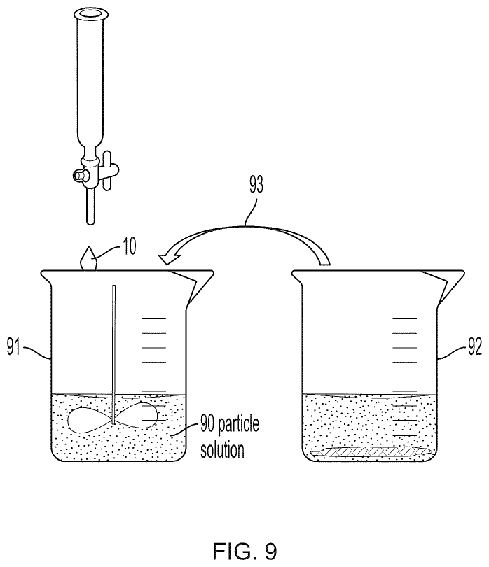

The present hybrid particles may be used to facilitate generation of substantially monoclonal clusters. Among other things, each of the present hybrid particles may include a scaffold molecule including single capture primer that may be used to capture a single target polynucleotide (e.g., a single single-stranded polynucleotide, or a single double-stranded polynucleotide). Additionally, each of the present hybrid particles may include a nanoparticle to which the scaffold molecule is coupled, and which includes amplification primers for use in amplifying the polynucleotide which is captured by the capture primer of the scaffold molecule to which the nanoparticle is coupled. The nanoparticle may have a diameter which substantially matches the size of a well into which the hybrid particle respectively becomes disposed, such that the hybrid particle sterically inhibits any other hybrid particle from being disposed within that well. As such, the single captured target polynucleotide may be amplified within the well so as to generate a substantially monoclonal cluster. In certain examples which will now be described with reference to FIGS. 1A-6C, the hybrid particle may include a dendritic molecule including include functional groups that can be used to covalently and/or non-covalently couple the dendritic molecule to the nanoparticle, and the polynucleotide may be hybridized to the capture primer before or after the hybrid particle is disposed in the well. Other examples, which may include scaffold molecules other than dendritic molecules, will be described further below with reference to FIGS. 7A-8B. Example operations for making the present hybrid molecules are described with reference to FIGS. 9-11B.

FIGS. 1A-1D schematically illustrate example operations for forming a hybrid particle and capturing a polynucleotide using the hybrid particle. Referring first to FIG. 1A, scaffold molecule 10 may include seeding primer 12 and a plurality of first moieties 16, and nanoparticle 17 may include a plurality of second moieties 18. The scaffold molecule 10 may be coupled to the nanoparticle 17 via interactions between the plurality of first moieties 16 and the plurality of second moieties 18. Illustratively, scaffold molecule 10 may be or include a dendritic molecule 10, which in the nonlimiting example shown in FIG. 1A may include dendritic core 11; seeding primer 12 coupled to the dendritic core; and a plurality of dendrons 13. Each of dendrons 13 includes an elongated polymer (illustratively, a polynucleotide, polypeptide, or synthetic polymer) that includes a first end 14 coupled to the dendritic core 11 and a second end 15 coupled to a functional group 16.

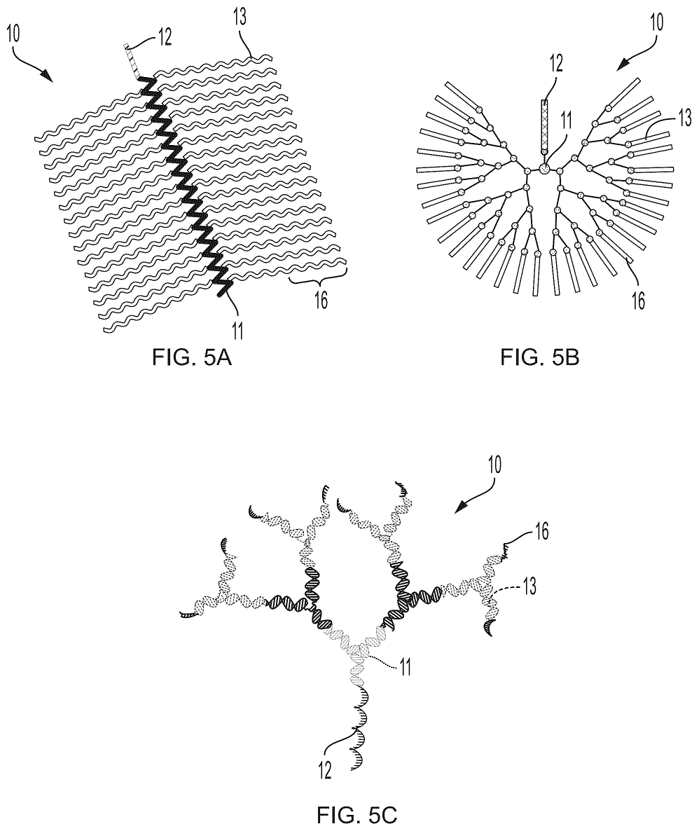

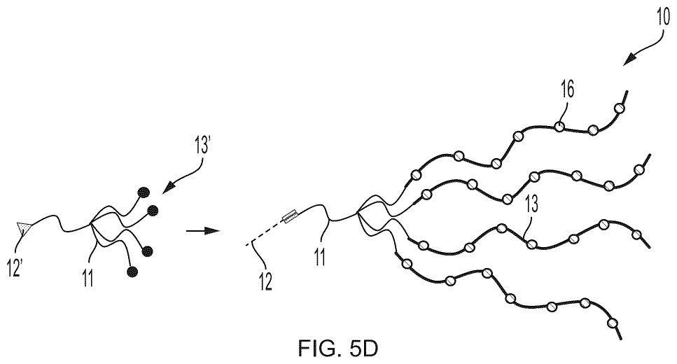

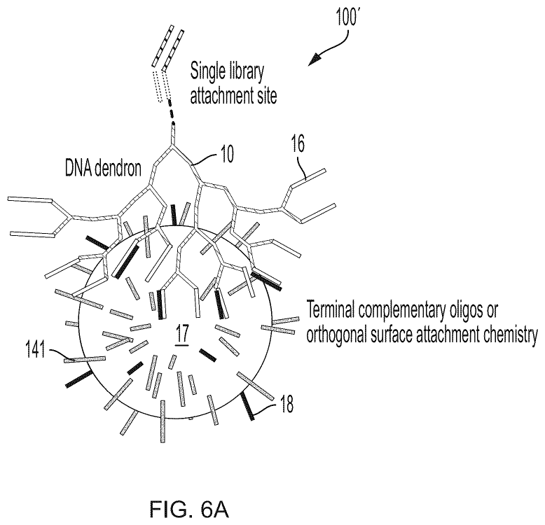

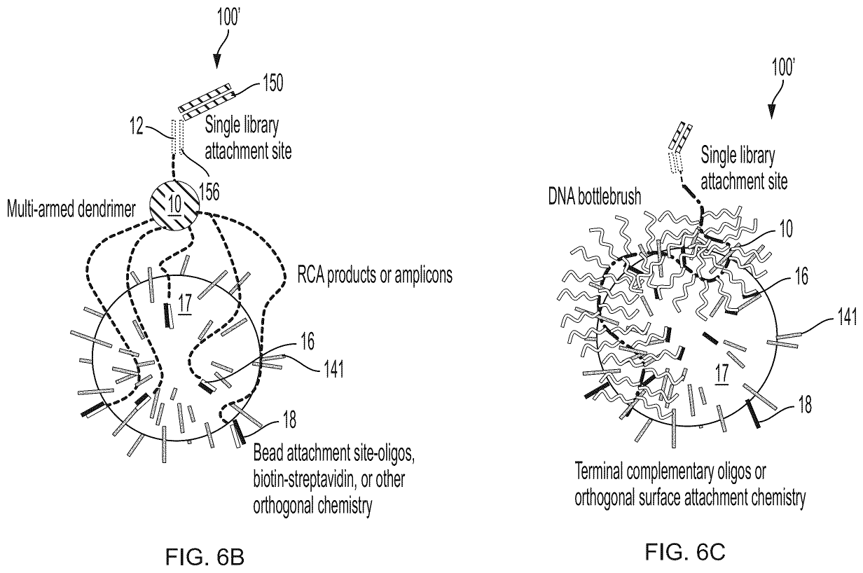

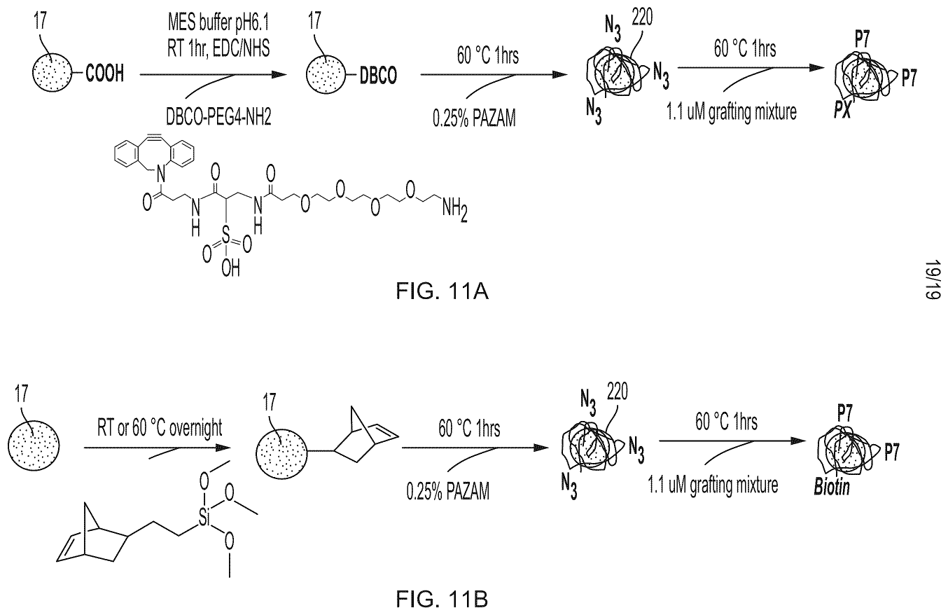

Nanoparticle 17 may include functional groups 18 that can react with respective functional groups 16 to form a covalent or non-covalent bond coupling the dendritic molecule 10 to nanoparticle 10 and thus forming hybrid particle 100, e.g., in a manner such as illustrated in FIG. 1B. Other nonlimiting examples of configurations of scaffold molecule 10 are described with reference to FIGS. 5A-5C and 7A-7B, and other nonlimiting examples of configurations of hybrid particle 100 are described with reference to FIGS. 6A-6C and 8A-8B. In some examples such as will be described with reference to FIGS. 11A-11B, nanoparticle 17 may include a hydrogel to which second moieties 18 are coupled. Optionally, nanoparticle 17 also may include amplification primers that may be used to amplify the captured polynucleotide, e.g., a single type of amplification primer 141 in the nonlimiting example shown in FIGS. 1A-1B, or a mixture of different types of amplification primers (e.g., 131 and 141). In examples in which the nanoparticle includes a hydrogel, the amplification primers (e.g., 141) may be coupled to the hydrogel (hydrogel not specifically illustrated in FIGS. 1A-1D). In one nonlimiting example, amplification primers 131 may include P5, and amplification primers 141 may include P7. In another nonlimiting example, amplification primers 141 may include P5, and amplification primers 131 may include P7.

As illustrated in FIG. 1C, hybrid particle 100 may be contacted with a fluid which may include target polynucleotides having a variety of lengths, e.g., polynucleotide fragments that are generated using commercially available fragmentation or tagmentation techniques, using DNA or RNA that it is desired to sequence. Illustratively, the hybrid particle may be contacted with a fluid including polynucleotide 150. Although only one polynucleotide is illustrated in FIG. 1C, it will be appreciated that the fluid may include thousands, or even millions, of fragments. The polynucleotides within the fluid may be substantially double-stranded. For example, polynucleotide 150 may include strand 151 which is hybridized to substantially complementary strand 151′.

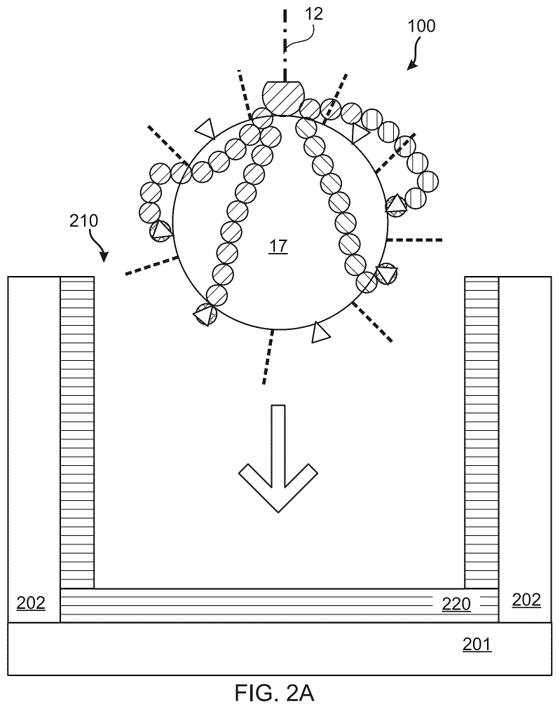

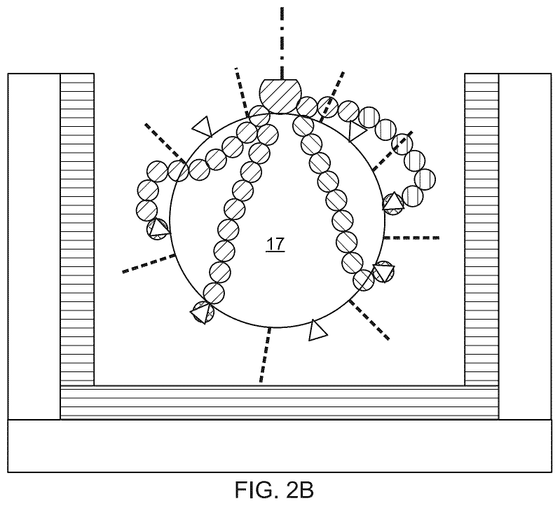

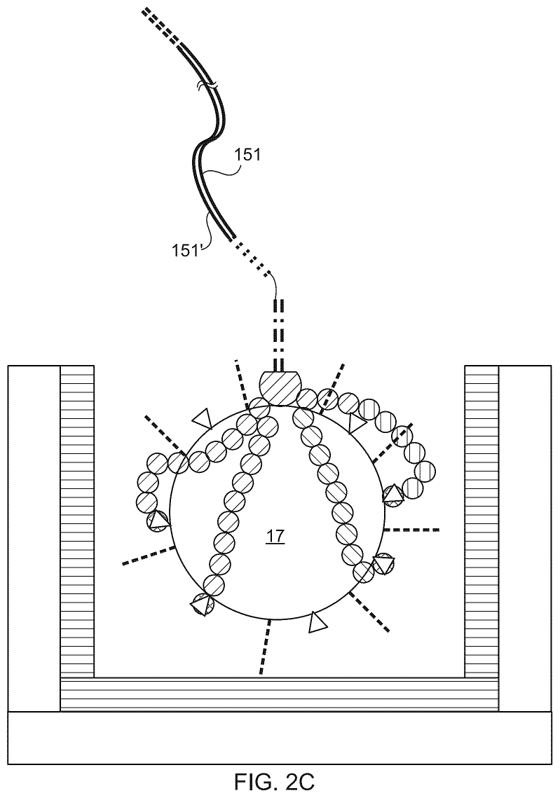

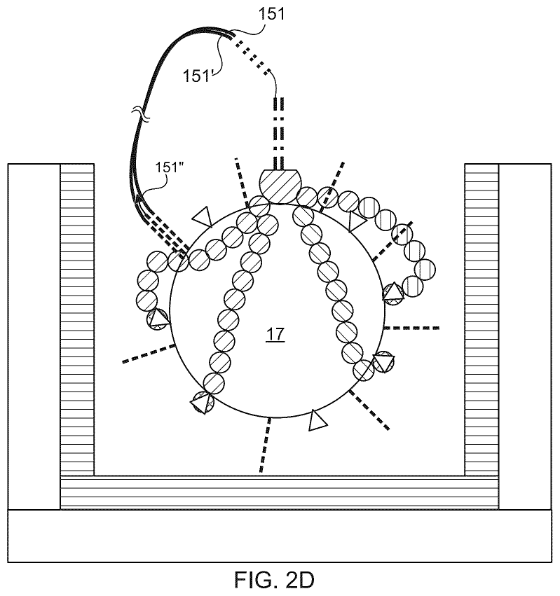

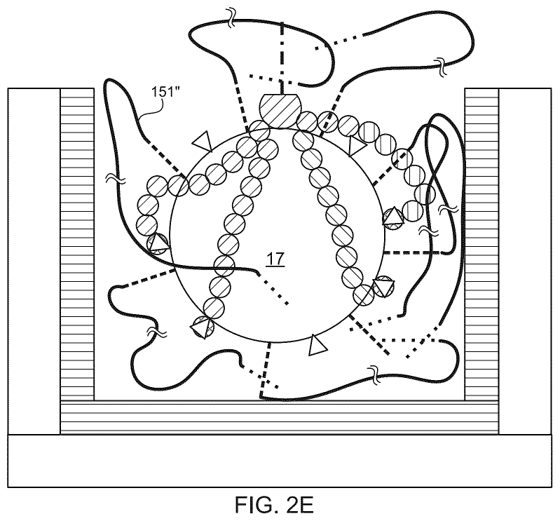

Strand 151 of polynucleotide 150 may include first and second adapters, e.g., first adapter 154 and second adapter 155 which may be used in a manner such as described with reference to FIGS. 2D-2E, and complementary strand 151′ may include adapter sequences that are complementary to 154 and 155, respectively; as such, these polynucleotides and their adapters may be referred to herein as being “double-stranded.” Additionally, in the example illustrated in FIG. 1C, polynucleotide 150 optionally may further include one or more additional adapters, e.g., seeding adapter 156 which may be coupled to adapter 154 and/or adapter 155, for example via a respective linker 158. However, note that while seeding adapter 156 may be coupled to strand 151, the complementary strand 151′ may not be coupled to the complements of such seeding adapter. Accordingly, the seeding adapter 156 may be single-stranded, and thus available to hybridize with a substantially complementary seeding primer 12 in a manner such as will be described with reference to FIG. 1D. In comparison, adapters 154 and 155 are double-stranded and thus unavailable to hybridize with amplification primers on the substrate until after certain processing steps are performed, as will be explained further below with reference to FIGS. 2A-2E. While the polynucleotide 150 (and adapters 154, 155 thereof) are illustrated as being double-stranded in FIGS. 1C-1D, in other examples herein the polynucleotides may be single-stranded, and their adapters may be single stranded, depending on the example.

In some examples, in a manner such as illustrated in FIG. 1D, hybrid particle 100 may be modified to generate modified hybrid particle 100′ including polynucleotide 150. More specifically, seeding adapter 156 of polynucleotide 150 may be hybridized to the seeding primer 12 of hybrid particle 100, to form modified hybrid particle 100′. Optionally, seeding primer 12 may have the sequence PX, and seeding adapter 156 may have the sequence cPX. However, it will be appreciated that seeding primer 12 and seeding adapter 156 may have any suitable sequences which are sufficiently complementary to one another to hybridize to one another. Such hybridization may be performed while hybrid particle 100 is in solution, e.g., suspended in the same fluid as is polynucleotide 150. Alternatively, such hybridization may be performed while hybrid particle 100 is disposed in a well of a flowcell, e.g., in a manner which will be described with reference to FIGS. 2A-2E.

Note that the operations of FIGS. 1A-1B for forming the hybrid particle 100 may be performed at any suitable time, and by any suitable entity, relative to the operations of FIGS. 1C-1D. For example, the operations of FIGS. 1A-1B for forming the hybrid particle 100 may be performed by a first entity, such as a manufacturer. The hybrid particles 100 then may be shipped to a second entity, such as a customer, that performs the operations of FIGS. 1C-1D using the hybrid particles that it receives from the first entity.

FIGS. 2A-2E schematically illustrate example operations for capturing and amplifying a polynucleotide using the hybrid particle 100 of FIG. 1B. Referring now to FIG. 2A, a plurality of hybrid particles 100 may be flowed into a flowcell that includes a plurality of wells 210. For example, the hybrid particle 100 which is illustrated in FIG. 2A may be one of a plurality of hybrid particles that are suspended in a fluid. In a manner such as described with reference to FIGS. 1A-1B, each hybrid particle 100 of the plurality may include a scaffold molecule 10 and a nanoparticle 17 that is coupled to the scaffold molecule 10. In the nonlimiting example illustrated in FIG. 2A, scaffold molecule 10 is a dendritic molecule 10 that includes a dendritic core 11; a seeding primer 12 coupled to the dendritic core; and a plurality of dendrons 12, each of the dendrons including an elongated polymer including a first end 14 coupled to the dendritic core and a second end 15 including a second functional group 16. Other nonlimiting examples of configurations of scaffold molecule 10 are described with reference to FIGS. 5A-5C and 7A-7B, and other nonlimiting examples of configurations of hybrid particle 100 are described with reference to FIGS. 6A-6C and 8A-8B.

The fluid containing the hybrid particles 100 may be flowed into a flowcell that includes well 210 which is one of a plurality of wells within substrate 200. Wells 210 may include a volume which is bounded, for example, by a bottom substrate 201 and one or more vertical sidewalls 202. The volume may be generated in any suitable manner, for example using nanoimprint lithography, conventional photolithography techniques, or the like. In the nonlimiting example illustrated in FIG. 2A, a hydrogel 220 may be disposed within respective wells 210.

Within at least some of the wells 210, a respective one of the hybrid particles 100 may become disposed within that well. For example, as intended to be represented in FIG. 2A by the large downward-pointing arrow, hybrid particle 100 may become inserted into well 210. For example, diffusion may transport the hybrid particle 100 to well 210, and then interactions between the hybrid particle and hydrogel 220 within the well may retain the hybrid particle within the well as illustrated in FIG. 2B. In examples in which wells 210 include hydrogel 220, hybrid particle 100 may covalently, non-covalently, and/or electrostatically interact with hydrogel 220 to retain the hybrid particle within well 210. Illustratively, forming a cluster of amplicons on hybrid particle 100 in a manner such as described below imparts a negative charge to the hybrid particle. Hydrogel 220 may be positively charged, for example by using a positively charged hydrogel. In examples in which wells 210 do not include hydrogel 220, hybrid particle 100 may covalently, non-covalently, and/or electrostatically interact directly with well 210 to retain the hybrid particle within well 210. For example, hydrophobic interactions between hybrid particle 100 and well 210 may retain the particle in the well. Additionally, or alternatively, electrostatic interactions between hybrid particle 100 and well 210 may retain the particle in the well. Illustratively, forming a cluster of amplicons on hybrid particle 100 in a manner such as described below imparts a negative charge to the hybrid particle. Well 210 may be positively charged, for example using silanization. Additionally, or alternatively, well 210 may include functional groups that are coupled to excess functional groups of hybrid particle 100. In examples in which hybrid particle 100 is reversibly (e.g., non-covalently) retained within well 210, the hybrid particle 100 optionally may be removed from the well after sequencing is complete, and the well 210 reused within another hybrid particle 100.

Additionally, as intended to be illustrated within FIGS. 2A-2B, hybrid particle 100 may have a diameter that sterically inhibits other hybrid particles from entering the same well 210. As such, well 210 (and other wells within the flowcell) may receive substantially a single one of the hybrid particles 100. For example, each of the hybrid particles 100 may have a diameter which is about 60% to about 100% of a diameter of wells 210. In examples in which hybrid particles 100 are not spherical (e.g., are oval, rod shaped, dumb-bell shaped, or the like), although the term “diameter” may not fully describe all dimensions of the hybrid particles, it should be understood that the dimensions of the hybrid particles suitably may be selected such that only a single particle fits within a given well 210, to the exclusion of other hybrid particles, so as to dispose a single seeding primer 12 at that well.

In some examples, after the hybrid particle 100 becomes coupled within the well 210, plurality of polynucleotides are flowed into the flowcell. The polynucleotides may be configured substantially as described with reference to FIGS. 1C-1D, e.g., respectively may include seeding adapters 156 that can hybridize to (e.g., are complementary to) seeding primers 12 of scaffold molecules 10, e.g., dendritic molecules. As illustrated in FIG. 2C, for each well in which a respective one of the hybrid particles 100 is disposed, a seeding adapter 156 of one of the polynucleotides 150 may hybridize to the seeding primer 12 of that hybrid particle. In some examples, the amplification primers 141 (and 131, if present) coupled to nanoparticle 17 and disposed within wells 210 may have a different sequence than the seeding primers 12 of hybrid particles 100, e.g., may be orthogonal to the seeding primers. As such, although polynucleotides 150 may come into contact with amplification primers 131 and/or 141 while being flowed through the flowcell, seeding adapters 156 may be substantially unable to hybridize to such amplification primers because they are not complementary. Additionally, during operations such as illustrated in FIG. 2C, seeding adapters 154, 155 of polynucleotides 150 may be substantially unable to hybridize to amplification primers 131 and/or 141 because they are double-stranded. Accordingly, capture of polynucleotides 150 are substantially limited to that by seeding primers 12. Additionally, because a single seeding primer is expected to be within each well 210, because a single hybrid particle can fit within each well, a single polynucleotide 150 is expected to be captured at each well.

For each well in which a respective one of the hybrid particles is disposed and for which the seeding adapter of one of the polynucleotides is hybridized to the seeding primer of that hybrid particle, the amplification primers 131 and/or 141 may be used to generate a substantially monoclonal cluster of amplicons of the polynucleotide hybridized to the seeding primer of that molecule. For example, as illustrated in FIG. 2D, double-stranded adapter 155 of captured polynucleotide 150 hybridizes with one of primers 141 to form a triplex using a process that may be referred to as “strand invasion” and may be promoted using a recombinase (not specifically illustrated in FIG. 2D). An amplified cluster then may be formed for the polynucleotide which is captured within each respective well 210. For example, FIG. 2D illustrates recombinase-promoted extension of the primer 141 to which double-stranded adapter 155 hybridizes to form amplicon 151″ which is covalently coupled to nanoparticle 17. Amplicon 151″ repeatedly may be further amplified using strand invasion. For example, it may be seen that the composition of FIG. 2E includes a plurality of amplicons that are formed using primers 141. In some examples (not specifically illustrated), the amplification primers may include excision moieties which may be used to remove amplicons that are oriented in a selected direction, so that the remaining amplicons are oriented substantially in the same direction as one another. In some examples, in wells 210 within substantially a single polynucleotide 150 is captured using a respective hybrid particle 100 and amplified using the amplification primers of that hybrid particle, the cluster of amplicons within that respective well 210 may be expected to be substantially monoclonal. Note that during amplification, the original polynucleotide 150 which was captured optionally may become dehybridized from seeding primer 12.

For further details regarding seeding and amplification operations using strand invasion and seeding adapters and seeding primers that are orthogonal to capture adapters and capture primers, see International Patent Publication No. WO2023/114394 entitled “Orthogonal Hybridization,” the entire contents of which are incorporated by reference herein.

As noted above with reference to FIG. 1A, linker 158 may be provided between adapter 154 and seeding adapter 156. In some examples, linker 158 may be or include one or more of: a carbon-containing chain with a formula (CH₂)ₙ wherein “n” is from 1 to about 1500, for example less than about 1000, preferably less than 100, e.g. from 2-50, particularly 5-25; polyethylene glycol (PEG); or iSp9 (Spacer 9) which is a triethylene glycol spacer that can be incorporated at the 5′-end or 3′-end of an oligo or internally. Linkers formed primarily from chains of carbon atoms or from PEG may be modified so as to contain functional groups which interrupt the chains. Examples of such groups include ketones, esters, amines, amides, ethers, thioethers, sulfoxides, sulfones. Separately or in combination with the presence of such functional groups, groups such as alkene, alkyne, aromatic or heteroaromatic moieties, or cyclic aliphatic moieties (e.g., cyclohexyl) may be included. Cyclohexyl or phenyl rings may, for example, be connected to a PEG or (CH₂)ₙ chain through their 1- and 4-positions. Other linkers are envisaged which are based on nucleic acids or monosaccharide units (e.g., dextrose). It is also within the scope of this disclosure to utilise peptides as linkers. A variety of other linkers may be employed. The linker preferably is stable under conditions under which the polynucleotides are intended to be used subsequently, e.g., conditions used in DNA amplification. The linker should also be such that it is not by-passed by DNA polymerases, terminating DNA polymerization before copying the seeding adapter 156 sequence (if it is nucleotide based such as a PX′ sequence). This allows for the seeding adapter to remain single-stranded and available for hybridization at all times.

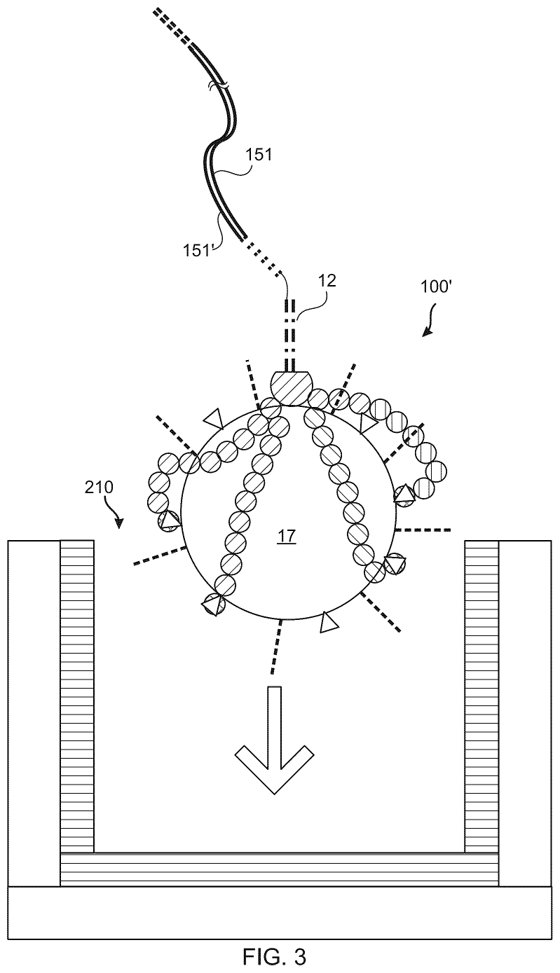

Note that FIGS. 2A-2E describe only one possible order of operations in which dendritic particles 100 may be used to capture and amplify respective polynucleotides 150. For example, FIG. 3 schematically illustrates an alternative example operation for use in capturing and amplifying a polynucleotide in the operations described with reference to FIGS. 2A-2E. In the example shown in FIG. 3, hybrid particle 100 captures polynucleotide 150 in solution in a manner such as described with reference to FIGS. 1C-1D to form modified hybrid particle 100′. The modified hybrid particle 100′ including polynucleotide 150 is then disposed within a corresponding well 210. The captured polynucleotide 150 then may be amplified in a manner such as described with reference to FIGS. 2D-2E. Note that in these examples, seeding primer 12 need not necessarily be orthogonal to amplification primers 131 and/or 141, because polynucleotide capture is performed off of the flow cell such that seeding adapter 156 substantially may not have the opportunity to hybridize to the amplification primers.

Note that the operations of FIGS. 2A-2B for disposing the hybrid particle 100 in well 210 may be performed at any suitable time, and by any suitable entity, relative to the operations of FIGS. 2C-2E. For example, the operations of FIGS. 2A-2B for disposing hybrid particles 100 in wells 210 may be performed by a first entity, such as a manufacturer. The wells 210 with hybrid particles 100 disposed therein then may be shipped to a second entity, such as a customer, that performs the operations of FIGS. 2C-2E using the hybrid particles and wells that it receives from the first entity.

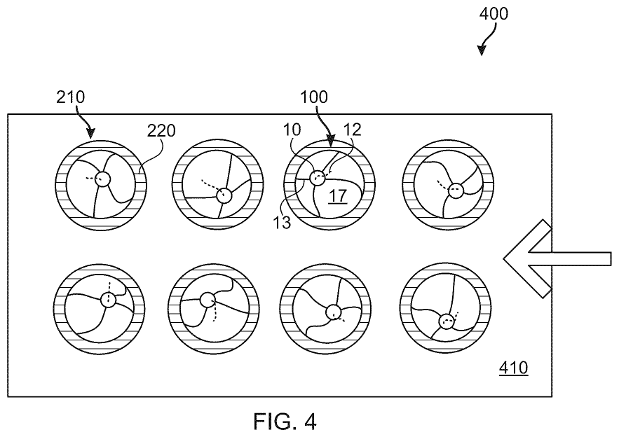

FIG. 4 schematically illustrates a device including a plurality of wells in which operations such as described with reference to FIGS. 2A-2E and/or 3 are implemented. Device 400 includes a flowcell 410 including a plurality of wells 210 such as described with reference to FIG. 2A, each well including a hydrogel; and a plurality of hybrid particles 100 such as described with reference to FIGS. 1A-1B, e.g., including scaffold molecules 10 including respective seeding primers 12 and coupled to respective nanoparticles 17. The hybrid particles 100 may be suspended in a fluid which is flowed into the flowcell in a manner as intended to be suggested by the large arrow, and once disposed in a respective well the hybrid particles 100 become disposed within respective wells 210. As illustrated in FIG. 4, at least some of the wells 210 (and optionally a majority of the wells, or more than 90% of the wells, or even substantially all of the wells) contain a single one of the hybrid particles 100. In some examples, the device may include polynucleotide(s) 150 which include a seeding adapter hybridized to the seeding primer, e.g., as described with reference to FIGS. 2C and 3. Similarly as intended to be represented by the large arrow in FIG. 4, the polynucleotides may be flowed into the flowcell and there may hybridize to the seeding primers of respective hybrid particles 100 within respective wells 210. The polynucleotide(s) may be double-stranded, and the seeding adapter may be single-stranded, e.g., as described with reference to FIGS. 1C-1D, although it will be appreciated that single-stranded polynucleotides alternatively may be used. In some examples, the device further may include, within wells 210 in which a respective one of the hybrid particles 100 is disposed and for which the seeding adapter 156 of one of the polynucleotides is hybridized to the seeding primer 12 of that hybrid particle, a substantially monoclonal cluster of amplicons, e.g., as described with reference to FIGS. 2D-2E.

Further details regarding example configurations of hybrid particles, example scaffold molecules suitable for use in hybrid particles, and example methods of preparing hybrid particles, now will be provided.

Although FIGS. 1A-1D, 2A-2E, 3, and 4 illustrate examples in which scaffold molecule 10 is a dendritic molecule, it will be appreciated that the present hybrid particles may include any suitable type of scaffold molecule. FIGS. 5A-5D schematically illustrate different types of dendritic molecules that may be used in the present hybrid particles. In the nonlimiting example illustrated in FIG. 5A, scaffold molecule 10 is a bottlebrush molecule that includes dendritic core 11, seeding primer 12, and dendrons 13. Dendritic core 11 is elongated and unbranched, and unbranched dendrons 13 extend outwardly from the dendritic core. For further details regarding bottlebrush molecules, see International Patent Publication No. WO2022/220748 entitled “Synthesis of polynucleotide bottlebrush polymer,” the entire contents of which are incorporated by reference herein. In the nonlimiting example illustrated in FIG. 5B, scaffold molecule 10 is a dendritic molecule that includes branched dendritic core 11, seeding primer 12, and branched dendrons 13, in which the dendritic core and dendrons include any suitable polymer(s) such as a synthetic polymer or polypeptide. In the nonlimiting example illustrated in FIG. 5C, scaffold molecule 10 is a dendritic molecule that includes branched dendritic core 11, seeding primer 12, and branched dendrons 13, in which the dendritic core and dendrons include DNA. For further details regarding dendritic molecules suitable for use in the present hybrid particles, see International Patent Publication No. WO2021/133770 entitled “Nanoparticle with Single Site for Template Polynucleotide Attachment,” the entire contents of which are incorporated by reference herein.

It will be appreciated that dendritic molecules may include any suitable number of dendrons 13 (which may be branched or unbranched) coupled to the dendritic core 11 (which may be branched or unbranched). The dendrons and the seeding primer may be coupled to the dendritic core in any suitable manner. It will further be appreciated that the present scaffold molecules 10 (e.g., dendritic molecules) may include any suitable material or combination of materials. Illustratively, in the nonlimiting example illustrated in FIG. 5D, scaffold molecule 10 is a dendritic molecule that includes branched dendritic core 11, seeding primer 12, and unbranched dendrons 13, in which the dendritic core includes any suitable polymer(s) such as a synthetic polymer or polypeptide, and in which the dendrons 13 include respective polynucleotides, e.g., RCA products. As illustrated in FIG. 5D, scaffold molecule 10 may be formed using a dendritic core 11 that includes moiety 12′ to which seeding primer 12 is coupled using a reaction between a first pair of complementary binding partners, and moieties 13′ to which dendrons 13 respectively are coupled using a reaction between a second pair of complementary binding partners. A non-exclusive list of complementary binding partners that may be used to couple elements together (e.g., to couple a seeding primer to a dendritic core, or to couple a dendron to a dendritic core), is presented in Table 1:

TABLE 1

| | Example moiety 12′ on | |
|---|---|---|
| | dendritic core 11 or moiety of | |
| | seeding primer 12 or moiety | |
| | 13′ on dendritic core or moiety | Example moiety to couple with that |
| Bonding pair | of dendron 13 | moiety |
| amine-NHS | amine group, —NH₂ | N-Hydroxysuccinimide ester |
| | | 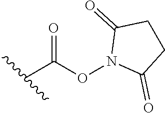 [SMILES: *C(=O)ON1C(=O)CCC1=O] |
| amine-imidoester | amine group, —NH₂ | imidoester |
| | | 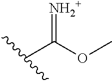 [SMILES: *C(=[NH2+])OC] |
| amine- | amine group, —NH₂ | pentafluorophenyl ester, |
| pentafluorophenyl ester | | |
| | | 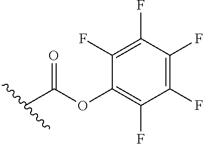 [SMILES: *C(=O)Oc1c(F)c(F)c(F)c(F)c1F] |
| amine-hydroxymethyl | amine group, —NH₂ | hydroxymethyl phosphine |
| phosphine | | |
| | | 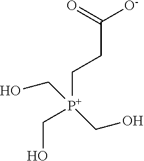 [SMILES: O=C([O-])CC[P+](CO)(CO)CO] |
| amine-carboxylic | amine group, —NH₂ | carboxylic acid group, —C(═O)OH (e.g., |
| acid | | following activation of the carboxylic |
| | | acid by a carbodiimide such as EDC (1- |
| | | ethyl-3-(-3-dimethylaminopropyl) |
| | | carbodiimide hydrochloride) or DCC |
| | | (N′,N′-dicyclohexyl carbodiimide) to |
| | | allow for formation of an amide bond |
| | | of the activated carboxylic acid with an |
| | | amine group) |
| thiol-maleimide | thiol, —SH | maleimide |
| | | 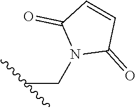 [SMILES: *CN1C(=O)C=CC1=O] |
| thiol-haloacetyl | thiol, —SH | haloacetyl (e.g., iodoacetyl or other haloacetyl) |
| | | 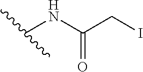 [SMILES: *NC(=O)CI] |
| thiol-pyridyl disulfide | thiol, —SH | pyridyl disulfide |
| | | 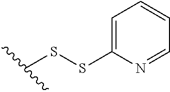 [SMILES: *SSc1ccccn1] |
| thiol-thiosulfonate | thiol, —SH | thiosulfonate |
| | | 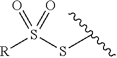 |
| thiol-vinyl sulfone | thiol, —SH | vinyl sulfone |
| | | 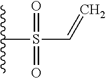 [SMILES: *S(=O)(=O)C=C] |
| aldehyde-hydrazide | aldehyde, —C(═O)H | hydrazide |
| | | 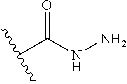 [SMILES: *C(=O)NN] |
| aldehyde-alkoxyamine | aldehyde, —C(═O)H | alkoxyamine |
| | | 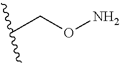 [SMILES: *CON] |
| hydroxy-isocyanate | hydroxyl, —OH | isocyanate |
| | | 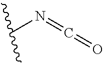 [SMILES: *N=C=O] |
| azide-alkyne | azide, —N₃ | alkyne |
| | | 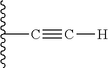 [SMILES: *C#C] |
| azide-phosphine | azide, —N₃ | phosphine, e.g .: |
| | | 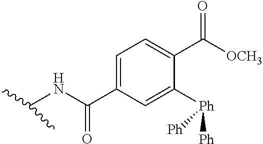 |
| azide-cyclooctyne | azide, —N₃ | cyclooctyne, e.g. dibenzocyclooctyne |
| | | (DBCO) |
| | | 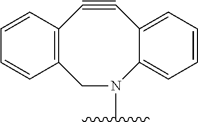 [SMILES: *N1Cc2ccccc2C#Cc2ccccc21] |
| | | or BCN (bicyclo[6.1.0]nonyne) |
| | | 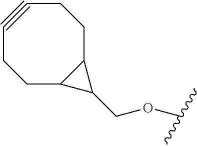 [SMILES: *OCC1C2CCC#CCCC21] |
| azide-norbornene | azide, —N₃ | norbornene |
| | | 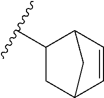 [SMILES: *C1CC2C=CC1C2] |
| transcyclooctene-tetrazine | transcyclooctene | tetrazine, e.g., benzyl-methyltetrazine |
| | 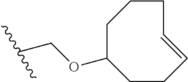 [SMILES: *COC1CC/C=C/CCC1] | 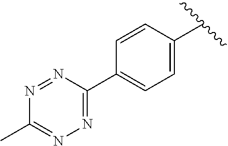 [SMILES: *c1ccc(-c2nnc(C)nn2)cc1] |
| norbornene-tetrazine | norbornene | tetrazine, e.g. benzyl-tetrazine |
| | 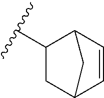 [SMILES: *C1CC2C=CC1C2] | 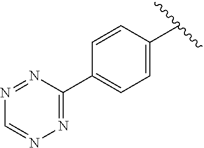 [SMILES: *c1ccc(-c2nncnn2)cc1] |
| oxime | aldehyde or ketone (e.g., amine | alkoxyamine |
| | group or N-terminus of | |
| | polypeptide converted to an | |
| | aldehyde or ketone by pyridoxal | |
| | phosphate) | |
| SpyTag- | SpyTag: amino acid sequence | SpyCatcher amino acid sequence: |
| SpyCatcher | AHIVMVDAYKPTK (SEQ ID | MKGSSHHHHHHVDIPTTENLYFQ |
| | NO: 9) | GAMVDTLSGLSSEQGQSGDMTIEE |
| | | DSATHIKFSKRDEDGKELAGATME |
| | | LRDSSGKTISTWISDGQVKDFYLY |
| | | PGKYTFVETAAPDGYEVATAITFT |
| | | VNEQGQVTVNGKATK (SEQ ID |
| | | NO: 10) |
| SNAP-tag-O⁶- | SNAP-tag (O-6-methylguanine- | O⁶-Benzylguanine |
| Benzylguanine | DNA methyltransferase) | |
| | | 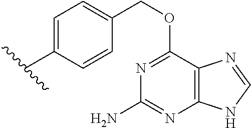 [SMILES: *c1ccc(COc2nc(N)nc3[nH]cnc23)cc1] |
| CLIP-tag-O²- | CLIP-tag (modified O-6- | O²-benzylcytosine |
| benzylcytosine | methylguanine-DNA methyltransferase) | |
| | | 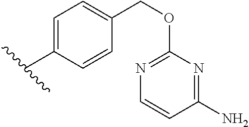 [SMILES: *c1ccc(COc2nccc(N)n2)cc1] |
| Sortase-coupling | -Leu-Pro-X-Thr-Gly (SEQ ID | -Gly₍₃₋₅₎ (SEQ ID NO: 12) (SEQ ID |
| | NO: 11) | NO: 13) |

FIGS. 6A-6C schematically illustrate example hybrid particles including different types of dendritic molecules. For example, FIG. 6A illustrates a hybrid particle including nanoparticle 17 and a DNA dendron 10 such as described with reference to FIG. 5C. FIG. 6B illustrates a hybrid particle including nanoparticle 17 and a multi-armed dendritic molecule (dendrimer) such as described with reference to FIG. 5B or 5D. FIG. 6C illustrates a hybrid particle including nanoparticle 17 and a bottlebrush dendron 10 such as described with reference to FIG. 5A. In the particular examples shown in FIGS. 6A-6C, the seeding primers 12 of the dendritic molecules 10 are hybridized to respective seeding adapters 156 of polynucleotides 150, and as such the hybrid particles are denoted 100′. However, it will be appreciated that the polynucleotides 150 may be coupled to the hybrid particles at any suitable time.

As noted further above, dendritic molecules are just one example of a scaffold molecule 10 that may be included in the present hybrid particles. Another nonlimiting example of a scaffold molecule 10 is a polynucleotide. FIGS. 7A-7B schematically illustrate different types of polynucleotides that may be used in the present hybrid particles. The polynucleotide may include a concatemer of repeating sequences. In the nonlimiting example illustrated in FIG. 7A, scaffold molecule 10 is or includes an RCA product. For example, scaffold molecule 10 illustrated in FIG. 7A may be generated using RCA template 70, primer 71, dNTPs, and a suitable polymerase (dNTPs and polymerase not specifically illustrated). Element 72 illustrated in FIG. 7A is a template polynucleotide that may be captured using scaffold molecule 10, e.g., after scaffold molecule 10 is coupled to a corresponding nanoparticle 17. In the nonlimiting example illustrated in FIG. 7B, scaffold molecule 10 is or includes expression of a plasmid. For example, scaffold molecule 10 illustrated in FIG. 7B may be generated using denatured plasmid 75′, primer 71′, dNTPs, and a suitable polymerase (dNTPs and polymerase not specifically illustrated). Element 72′ in FIG. 7B is an optional dendron with clustering oligos that may be attached using click chemistry.

FIGS. 8A-8B schematically illustrate example hybrid particles including different types of polynucleotides. For example, FIG. 8A illustrates a hybrid particle including nanoparticle 17 and polynucleotide 10 which is expression of a plasmid such as described with reference to FIG. 7B. In some examples of the configuration illustrated in FIG. 8A, the template polynucleotide 150 may hybridize to the capture oligo on the plasmid expression polynucleotide 10 and extends around the plasmid to generate the full plasmid sequence, and the branch is the capture oligo hybridized to the library as shown in FIG. 7B. FIG. 8B illustrates a hybrid particle including nanoparticle 17 and RCA product 10 such as described with reference to FIG. 7A. In the particular examples shown in FIGS. 6A-6C, the seeding primers 12 of the dendritic molecules 10 are hybridized to respective seeding adapters 156 of polynucleotides 150, and as such the hybrid particles are denoted 100′. However, it will be appreciated that the polynucleotides 150 may be coupled to the hybrid particles at any suitable time.

It will be appreciated that in the present hybrid particles 100, the scaffold molecule 10 may be coupled to the nanoparticle 17 in any suitable manner. That is, moiety 16 and moiety 18 described with reference to FIGS. 1A-1B may be or include any suitable complementary binding partners that may be used to couple scaffold molecule 10 to nanoparticle 17. In one nonlimiting example, moieties 16 and 18 may include any suitable pair of complementary binding partners in Table 1. In another nonlimiting example, moieties 16 and 18 couple to one another via a noncovalent interaction (which also may be referred to as noncovalent bonding). Illustratively, the noncovalent interaction may be avidin-biotin interaction. For example, first moiety 16 may include a biotin moiety that allows for non-covalent bonding with a respective streptavidin binding site located on second moiety 18. Or, for example, second moiety 18 may include a biotin moiety that allows for non-covalent bonding with a respective streptavidin binding site located on first moiety 16. A non-exclusive list of complementary binding partners that may be used to non-covalently bond moiety 16 to moiety 18 is presented in Table 2:

TABLE 2

| | Example moiety 16 | Example moiety 18 or |
|---|---|---|
| Bonding pair | or 18 | 16 |
| biotin-streptavidin | biotin, | Streptavidin, |
| | desthiobiotin, dual- | neutravidin, avidin, |
| | biotin, Strep-tag | Strep-Tactin |
| His-tag transition | transition metal | Histidine tag (His-tag) |
| metal | (e.g., Mn²⁺, Fe²⁺, | |
| | Co₂₊, Ni²⁺, or Cu²⁺) | |
| His-tag transition | His-tag | transition metal (e.g., |
| metal | | Mn²⁺, Fe²⁺, Co₂₊, Ni²⁺, |
| | | or Cu²⁺) |
| DIG/anti-DIG | digoxigenin (DIG) | anti-digoxigenin (anti- |
| | | DIG) antibody |
| c-myc/anti-cmyc | c-myc (also referred | anti-cmyc antibody |
| | to as MYC) | |
| c-myc/anti-cmyc | anti-cmyc antibody | c-myc |
| GST/glutathione | glutathione | glutathione s-transferase |
| | | (GST) |
| GST/glutathione | glutathione s- | glutathione |
| | transferase (GST) | |
| FLAG/anti-FLAG | FLAG tag | Anti-FLAG antibody |
| FLAG/anti-FLAG | Anti-FLAG | FLAG tag |
| | antibody | |

FIGS. 7A-7B illustrate examples in which moiety 16 of scaffold molecule 10 is a biotinylated dNTP that is incorporated into the RCA product or plasmid expression. Nanoparticle 17 may be functionalized to include streptavidin or other suitable moiety (e.g., from Table 2) to which the biotinylated dNTPs may bond so as to couple scaffold molecule 10 to nanoparticle 17. In the nonlimiting example illustrated in FIG. 7B, the scaffold molecule 10 and nanoparticle 17 (not specifically shown) may be disposed within a streptavidin-coated well 210 and retained there via interactions between the biotinylated dNTPs and streptavidin in the well.

As yet another example, moiety 16 may be or include a first oligonucleotide, and moiety 18 may be or include a second oligonucleotide to which the first oligonucleotide is hybridized to couple the scaffold molecule 10 to the nanoparticle 17. Seeding primer 12 may be orthogonal to the second oligonucleotide and/or may be orthogonal to the first oligonucleotide. FIGS. 5A-5C illustrate examples in which moiety 16 is a first oligonucleotide that constitutes at least a portion of a corresponding dendron 13. As illustrated in FIGS. 6A-6C, moieties 18 of nanoparticle 17 are or include second oligonucleotides that are complementary to, and thus hybridize to, the first oligonucleotides of moieties 16 of scaffold molecules 10. In these examples, moieties 16 may be located at the ends of respective dendrons 13. However, moieties 16 need not necessarily be at the end of dendrons. Illustratively, the moieties may be located along the length of one or more elements of scaffold molecule 10. For example, in the examples shown in FIGS. 8A-8B, the first oligonucleotides of moieties 16 occur repeatedly along the length of the elongated polynucleotide which is the plasmid expression (FIG. 8A) or RCA product (FIG. 8B), and respectively hybridize to different ones of the second oligonucleotides of moieties 18 of nanoparticle 17.

Hybrid particles 100 such as described with reference to FIGS. 1A-1D, 2A-2E, 3, 4, 5A-5D, 6A-6C, 7A-7B, and 8A-8B may be formed using any suitable combination and order of operations. Illustratively, a method of making a hybrid particle may include suspending a plurality of nanoparticles in a solution, wherein each of the nanoparticles includes a plurality of first moieties. FIG. 9 schematically illustrates an example flow of operations in a method for preparing hybrid particles. For example, nanoparticles 17 including moieties 18 (e.g., a moiety from Table 1, or from Table 2, or an oligonucleotide) may be suspended in a solution 90. In some examples, solution 90 is aqueous, organic, or mixed-phase. An aqueous solution 90 may be particularly suitable in examples in which DNA-DNA interactions are used to form hybrid particle 100.

The method may include adding a plurality of scaffold molecules to the solution, wherein each of the scaffold molecules includes a capture primer and a plurality of second moieties. For example, scaffold molecules 10 including capture primer (seeding primer) 12 and moieties 16 (e.g., a moiety from Table 1, or from Table 2, or an oligonucleotide that is complementary to an oligonucleotide of nanoparticles 17) may be added to the solution in which nanoparticles 17 are suspended. The method may include forming a first hybrid particle 100 by coupling one of the nanoparticles 17 to a corresponding one of the scaffold molecules 10 via interactions between the plurality of first moieties 18 of that nanoparticle and the plurality of second moieties 16 of that scaffold molecule. For example, complementary binding partners from Table 1 may covalently bond with one another, or complementary binding partners from Table 2 may noncovalently bond with one another, or complementary oligonucleotides may hybridize to one another, so as to couple scaffold molecules 17 to corresponding nanoparticles 17. Scaffold molecules 10 may be added to solution 90 at a rate and at a concentration at which nanoparticles are statistically likely to become coupled either to only a single one of scaffold molecules 10 or to none of scaffold molecules 10. Nonetheless, in some examples, some nanoparticles 17 may become coupled to two scaffold molecules or more than two scaffold molecules.

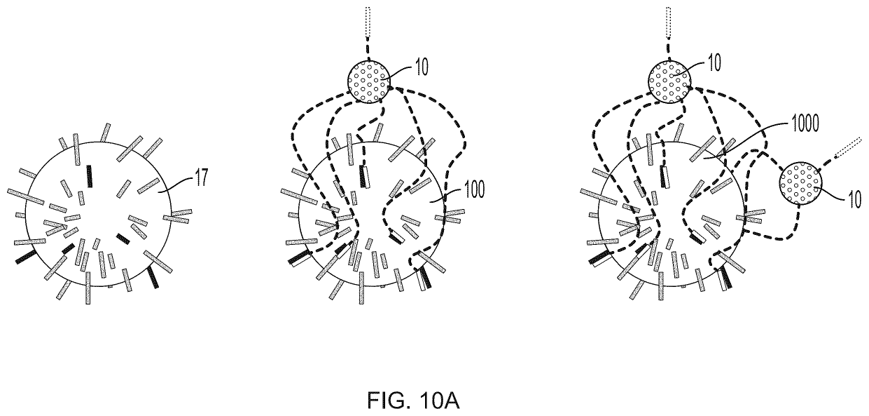

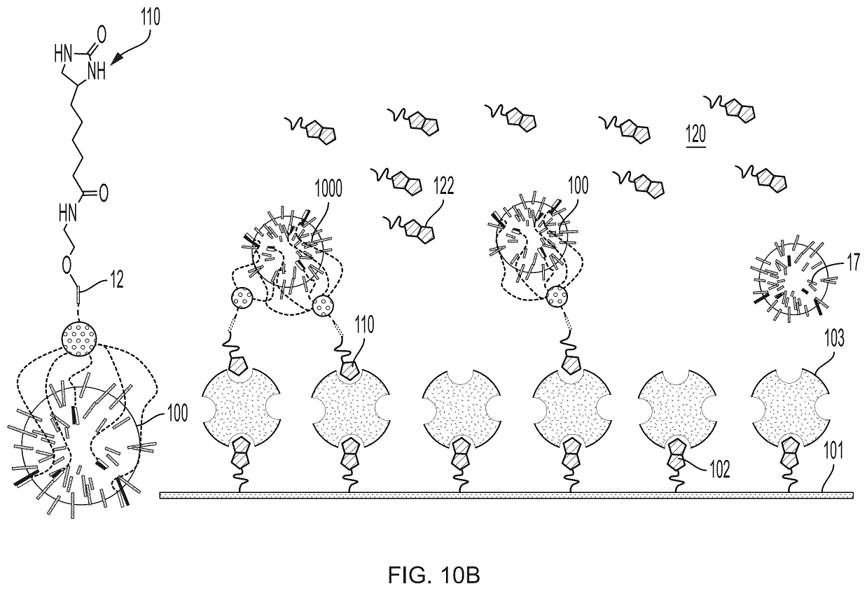

FIGS. 10A-10B schematically illustrate another example flow of operations in a method for preparing hybrid particles. As illustrated in FIG. 10A, hybrid particles 100 that include a single nanoparticle 17 coupled to a single scaffold molecule 10 may be separated in any suitable manner from nanoparticles 17 that are not coupled to any scaffold molecule. Illustratively, and referring again to FIG. 9, solution 90 optionally is located in a vessel 91 that includes a region to which a plurality of complementary capture primers are coupled (that is, sequences that are complementary to, and hybridize to, capture primer 12). The method further may include coupling hybrid particles 100 (e.g., the first hybrid particle mentioned above with reference to FIG. 9) to the region of the vessel 91 via hybridization between the capture primer 12 of the scaffold molecule 10 of that hybrid particle and a corresponding one of the complementary capture primers. While the first hybrid particle is coupled to the region of the vessel, at least a portion of the solution may be removed (e.g., to vessel 92 illustrated in FIG. 9), wherein the removed portion contains nanoparticles that are not coupled to a corresponding one of the scaffold molecules and thus are not coupled to the region of the vessel 91. As intended to be represented in FIG. 9 by operation 93, additional scaffold molecules may be added to the removed solution (which solution optionally may be relocated back to vessel 91), and another hybrid particle may be formed by coupling one of the nanoparticles in the removed solution to a corresponding one of the additional scaffold molecules 10 via interactions between the plurality of first moieties of that nanoparticle and the plurality of second moieties of that scaffold molecule. The hybrid particles 100 coupled to the region of vessel 91 then may be eluted into another solution, e.g., by dehybridizing the capture primers 12 of the scaffold molecules 10 of those hybrid particles 100 from the complementary capture primers coupled to that region of the vessel, and then removing the hybrid particles 100 for subsequent use.

As also illustrated in FIG. 10A, hybrid particles 100 that include a single nanoparticle 17 coupled to a single scaffold molecule 10 also or alternatively may be separated in any suitable manner from second hybrid particles 1000 that include a nanoparticle coupled to more than one scaffold molecule (e.g., two scaffold molecules, or more than two scaffold molecules), that is formed by coupling one of the nanoparticles 17 to a corresponding two or more of the scaffold molecules 10 via interactions between the moieties 18 of that nanoparticle and the moieties 16 of those scaffold molecules. For example, the first hybrid particle 10 and the second hybrid particle 1000 may be coupled to a surface, wherein the second hybrid particle 1000 couples to the surface with a different force than does the first hybrid particle 100. The different force may be used to separate the first hybrid particle 100 from the second hybrid particle 1000. In the illustrative example shown in FIG. 10B, surface 101 (which may be glass, silica, or another suitable material) includes biotin moieties 102 that are coupled to respective streptavidin molecules 103. Capture primers 12 of respective scaffold molecules 10 respectively may be coupled to desthiobiotin 110 or other biotin analogue that binds more weakly to streptavidin than does biotin. The desthiobiotin 110 of particles 100 and 1000 binds to the streptavidin molecules 103. Because particle 100 includes a single desthiobiotin 110 and particle 1000 includes two desthiobiotins 110, particle 100 couples to surface 101 with a different force than does particle 1000. More specifically, particle 100 binds more weakly to surface 101 than does particle 1000. In contrast to particles 100 and 1000, nanoparticle 17 which is not coupled to a scaffold molecule 10 does not bind to surface 101 and can be washed away while particles 100 and 100 are coupled to the surface.

The difference in binding strength between particle 100 and particle 1000 can be used to separate these two types of particles from one another. For example, as shown in FIG. 10B, a solution 120 including biotin 122 may be flowed over surface 101. Similar to column chromatography, this can be performed starting at a low concentration and gradually increasing the biotin concentration flowing over the surface. Biotin 122 binds more tightly to streptavidin 130 at the surface 101 than does desthiobiotin 110. Therefore, biotin 122 may have a concentration in solution 120 that is selected to displace the more weakly bound hybrid particles (that is, hybrid particles 100) while the more strongly bound hybrid particles (that is, hybrid particles 1000) remain bound to surface 101. By controlling the concentration of biotin 122 flowing over the surface 101, the release of hybrid particles can be controlled such that a substantially pure fraction of hybrid particles 100 (including a single nanoparticle 17 and a single scaffold molecule 10) may be separated and collected.

As noted further above, in some examples nanoparticles 17 may include a hydrogel coating. This hydrogel may be disposed over (and may substantially surround) a hard core particle that has a composition other than a hydrogel, e.g., a rigid polymer (such as polyethylene, polystyrene, or polypropylene), a glass (such as silica), or a magnetic material. The surface of the hard core particle may be functionalized so as to be covalently or non-covalently coupled to the hydrogel. Illustratively, the hard core particle may be functionalized by one or more of silanization, thiolation, surface polymerization, polymer grafting, polymer adsorption, peptide conjugation, or protein conjugation, or any combination thereof, to introduce one or more functional groups such as azide, carboxyl, NHS, amine, aldehyde, epoxy, isothiocyanate, maleimide, or the like (e.g., functional groups in Table 1). The hydrogel including moieties 18 and amplification primers 131 and/or 141 may be coupled to the functionalized hard core particle in any suitable manner, e.g., in a manner such as described with reference to FIGS. 11A-11B. Alternatively, moieties 18 and amplification primers 131 and/or 141 may be coupled directly to the functionalized hard core particle without use of a hydrogel.

FIGS. 11A-11B schematically additional example flows of operations in a method for preparing hybrid particles. More specifically, FIG. 11A illustrates an example in which a magnetic nanoparticle 17 is functionalized to include —COOH groups. The —COOH groups are coupled to respective alkyne groups (here, DBCO) using a molecule such as DBCO-PEG4-NH₂, where the —COOH and —NH₂ form an amide bond linking the DBCO to the magnetic nanoparticle 17. In the illustrated example, the reaction is performed in a buffer solution using EDC/NHS to activate the reaction. Hydrogel 220 including —N₃ groups (illustratively, PAZAM) then is coupled to the magnetic particle 17 via reaction between the —N₃ groups of the hydrogel and the DBCO. Amplification primers (e.g., P7) and moieties 18 (e.g., PX) then are coupled to the —N₃ groups of the hydrogel. FIG. 11B illustrates an example in which a silica nanoparticle 17 is silanized to include norbornene groups. Hydrogel 220 including —N₃ groups (illustratively, PAZAM) then is coupled to the silica particle 17 via reaction between the —N₃ groups of the hydrogel and the norbornene. Amplification primers (e.g., P7) and moieties 18 (e.g., biotin) then are coupled to the —N₃ groups of the hydrogel.

From the foregoing disclosure, it will be appreciated that primers and adapters having any suitable sequences may be used. In one nonlimiting example, amplification primers 131 are P5 amplification primers, and the amplification primers 141 are P7 amplification primers. P5 amplification primers, which are commercially available from Illumina, Inc. (San Diego, CA) have the sequence 5′-AATGATACGGCGACCACCGA-3′ (SEQ ID NO: 1). P7 amplification primers, which also are commercially available from Illumina, Inc., have the sequence 5′-CAAGCAGAAGACGGCATACGA-3′ (SEQ ID NO: 2). Alternatively amplification primers 141 may be P7 and amplification primers 131 may be P5. Adapters 154 may be full-length complementary P5 adapters (cP5) having the sequence 5′-TCGGTGGTCGCCGTATCATT-3′ (SEQ ID NO: 3), and are commercially available from Illumina, Inc. Adapters 155 may be full-length complementary P7 adapters (cP7) having the sequence 5′-TCGTATGCCGTCTTCTGCTTG-3′ (SEQ ID NO: 4), and are commercially available from Illumina, Inc. Seeding primers 12 may have any suitable sequence which is orthogonal to the sequences of amplification primers 131, 141. In some examples, seeding primers 12 may be PX primers having the sequence AGGAGGAGGAGGAGGAGGAGGAGG (SEQ ID NO: 5), or PY primers having the sequence 5′-GAA GAA GAA GAA GAA GAA GAA GAA GAA GAA-3′ (SEQ ID NO: 6). Seeding adapters 156 may be cPX (also referred to as PX′) primers having the sequence CCTCCTCCTCCTCCTCCTCCTCCT (SEQ ID NO: 7) or cPY (PY′) primers having the sequence 5′-TTC TTC TTC TTC TTC TTC TTC TTC TTC TTC-3′ (SEQ ID NO: 8).

It will further be appreciated that various examples herein may be used with operations consistent with “bridge amplification” or “surface-bound polymerase chain reaction” and/or with other amplification modalities. One such amplification modality is “exclusion amplification,” or ExAmp. Exclusion amplification methods may allow for the amplification of a single target polynucleotide per substrate region and the production of a substantially monoclonal population of amplicons in a substrate region. For example, the rate of amplification of the first captured target polynucleotide within a substrate region may be more rapid relative to much slower rates of transport and capture of target polynucleotides at the substrate region. As such, the first target polynucleotide captured in a substrate region may be amplified rapidly and fill the entire substrate region, thus inhibiting the capture of additional target polynucleotide in the same substrate region. Alternatively, if a second target polynucleotide attaches to same substrate region after the first polynucleotide, the relatively rapid amplification of the first polynucleotide may fill enough of the substrate region to result in a signal that is sufficiently strong to perform sequencing by synthesis (e.g., the substrate region may be at least functionally monoclonal). The use of exclusion amplification may also result in super-Poisson distributions of monoclonal substrate regions; that is, the fraction of substrate regions in an array that are functionally monoclonal may exceed the fraction predicted by the Poisson distribution.

Increasing super-Poisson distributions of useful clusters is useful because more functionally monoclonal substrate regions may result in higher quality signal, and thus improved SBS; however, the seeding of target polynucleotides into substrate regions may follow a spatial Poisson distribution, where the trade-off for increasing the number of occupied substrate regions is increasing the number of polyclonal substrate regions. One method of obtaining higher super-Poisson distributions is to have seeding occur quickly, followed by a delay among the seeded target polynucleotide. The delay, termed “kinetic delay” because it is thought to arise through the biochemical reaction kinetics, gives one seeded target polynucleotide an earlier start over the other seeded targets. Exclusion amplification works by using recombinase to facilitate the invasion of primers (e.g., primers attached to a substrate region) into double-stranded DNA (e.g., a target polynucleotide) when the recombinase mediates a sequence match. The present compositions and methods may be adapted for use with recombinase to facilitate the invasion of the present capture primers and orthogonal capture primers into the present target polynucleotides when the recombinase mediates a sequence match. Indeed, the present compositions and methods may be adapted for use with any surface-based polynucleotide amplification methods such as thermal PCR, chemically denatured PCR, and enzymatically mediated methods (which may also be referred to as recombinase polymerase amplification (RPA), strand invasion, or ExAmp). For further details of amplification processes such as may be used during, and/or are compatible with, operations such as described with reference to FIGS. 2C-2D, see International Patent Application No. PCT/US2022/053005 to Ma et al., filed Dec. 15, 2022 and entitled “Hybrid Clustering,” and International Patent Application No. PCT/EP2023/058307 to Ma et al., filed Mar. 30, 2023 and entitled “Paired-End Resynthesis Using Blocked P5 Primers,” the entire contents of each of which are incorporated by reference herein.

### ADDITIONAL COMMENTS

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A hybrid particle for use in capturing a first polynucleotide, the hybrid particle comprising:
a scaffold molecule comprising a seeding primer and a plurality of first moieties; and
a nanoparticle comprising a plurality of second moieties,
wherein the scaffold molecule is coupled to the nanoparticle via interactions between the plurality of first moieties and the plurality of second moieties.

2. The hybrid particle of claim 1, wherein the scaffold molecule comprises a dendritic molecule comprising:
a dendritic core to which the seeding primer is coupled; and
a plurality of dendrons.

3. The hybrid particle of claim 2, wherein each of the dendrons comprises an elongated polymer.

4. The hybrid particle of claim 3, wherein the elongated polymer comprises a second polynucleotide.

5. The hybrid particle of claim 3, wherein the elongated polymer comprises an inert polymer.

6. The hybrid particle of claim 1, wherein the scaffold molecule comprises a second polynucleotide.

7. The hybrid particle of claim 6, wherein the second polynucleotide comprises a concatemer of repeating sequences.

8. The hybrid particle of claim 7, wherein the concatemer of repeating sequences comprises a rolling circle amplification (RCA) product or expression of a plasmid.

9. (canceled)

10. The hybrid particle of claim 1, wherein the scaffold molecule comprises a bottlebrush molecule.

11. The hybrid particle of claim 1, wherein the nanoparticle comprises a silica core.

12. The hybrid particle of claim 1, wherein the nanoparticle comprises a magnetic core.

13. The hybrid particle of claim 1, wherein the nanoparticle comprises a hydrogel to which the second moieties are coupled.

14. The hybrid particle of claim 13, further comprising a plurality of amplification primers coupled to the hydrogel.

15. (canceled)

16. (canceled)

17. The hybrid particle of claim 1, wherein each of the first moieties comprises a first oligonucleotide.

18. The hybrid particle of claim 17, wherein each of the second moieties comprises a second oligonucleotide to which the first oligonucleotide is hybridized to couple the scaffold molecule to the nanoparticle.

19. (canceled)

20. (canceled)

21. The hybrid particle of claim 1, wherein the first moiety and the second moiety are covalently bonded to couple the scaffold molecule to the nanoparticle.

22. The hybrid particle of claim 1, further comprising the first polynucleotide, wherein the first polynucleotide comprising a seeding adapter that is hybridized to the seeding primer.

23. The hybrid particle of claim 22, wherein the first polynucleotide is double-stranded, and wherein the seeding adapter is single-stranded.

24-31. (canceled)

32. A device, comprising:
a flowcell comprising a plurality of wells; and
a plurality of hybrid particles, each hybrid particle comprising:
a scaffold molecule comprising a seeding primer and a plurality of first moieties; and
a nanoparticle comprising a plurality of second moieties,
wherein the scaffold molecule is coupled to the nanoparticle via interactions between the plurality of first moieties and the plurality of second moieties;
wherein at least some of the wells contain a single one of the hybrid particles.

33. (canceled)

34. A method of capturing a polynucleotide in a flowcell, the method comprising:
flowing a plurality of hybrid particles into a flowcell comprising a plurality of wells, each hybrid particle comprising:
a scaffold molecule comprising a seeding primer and a plurality of first moieties; and
a nanoparticle comprising a plurality of second moieties,
wherein the scaffold molecule is coupled to the nanoparticle via interactions between the plurality of first moieties and the plurality of second moieties; and
within at least some of the wells, respectively disposing a respective one of the hybrid particles within that well.

35-41. (canceled)
